SEQUENCE LISTING (2 sequences)

SEQ ID NO: 1 - RNA, 21 nt, synthetic construct
  source 1..21: mol_type = other RNA
  modified_base 1: mod_base = OTHER; note = 2'-fluoroguanosine with phosphorothioate linkage
  modified_base 2: mod_base = OTHER; note = 2'-O-methylguanosine with phosphorothioate linkage
  modified_base 3: mod_base = OTHER; note = 2'-fluorouridine
  modified_base 4: mod_base = OTHER; note = 2'-O-methyluridine
  modified_base 5: mod_base = OTHER; note = 2'-fluoroadenosine
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = OTHER; note = 2'-O-methyluridine
  modified_base 13: mod_base = OTHER; note = 2'-fluorouridine
  modified_base 14: mod_base = OTHER; note = 2'-O-methyluridine
  modified_base 15: mod_base = OTHER; note = 2'-fluoroadenosine
  modified_base 16: mod_base = OTHER; note = 2'-O-methylcytidine
  modified_base 17: mod_base = OTHER; note = 2'-fluorouridine
  modified_base 18: mod_base = OTHER; note = 2'-O-methyluridine
  modified_base 19: mod_base = OTHER; note = 2'-fluorocytidine
  modified_base 20: mod_base = OTHER; note = 2'-O-methyladenosine
  modified_base 21: mod_base = OTHER; note = 2'-fluoroadenosine
ggttaacaccatttacttcaa

SEQ ID NO: 2 - RNA, 23 nt, synthetic construct
  source 1..23: mol_type = other RNA
  modified_base 1: mod_base = OTHER; note = 2'-O-methyluridine with phosphorothioate linkage
  modified_base 2: mod_base = OTHER; note = 2'-fluorouridine with phosphorothioate linkage
  modified_base 3: mod_base = OTHER; note = 2'-O-methylguanosine
  modified_base 4: mod_base = OTHER; note = 2'-fluoroadenosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyluridine
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyluridine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluorouridine
  modified_base 15: mod_base = OTHER; note = 2'-O-methylguanosine
  modified_base 16: mod_base = OTHER; note = 2'-fluorouridine
  modified_base 17: mod_base = OTHER; note = 2'-O-methyluridine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorocytidine
  modified_base 21: mod_base = OTHER; note = 2'-O-methylcytidine with phosphorothioate linkage
  modified_base 22: mod_base = OTHER; note = 2'-O-methyladenosine with phosphorothioate linkage
  modified_base 23: mod_base = OTHER; note = 2'-O-methylguanosine
ttgaagtaaatggtgttaaccag

(19) United States
(12) Patent Application Publication
(10) Pub. No.: US 2026/0185097 A1
(43) Pub. Date: Jul. 2, 2026

(54) TREATMENT OF HEMOPHILIA A WITH FITUSIRAN

(71) Applicant: Genzyme Corporation, Cambridge, MA (US)

(72) Inventors: Shauna ANDERSSON, Lexington, MA (US); Viridiana Cano, Mexico City (MX); Ekta Seth Chhabra, Westborough, MA (US); Sajida Iqbal, Brimingham (GB); Salim Kichou, Ia rivière de Corps (FR); Barbara Kittner, Bedminster, NJ (US); Laurel Menapace, Monrovia, MD (US)

(21) Appl. No.: 19/565,331

(22) Filed: Mar. 12, 2026

Related U.S. Application Data
(63) Continuation of application No. PCT/US2024/046873, filed on Sep. 16, 2024.
(60) Provisional application No. 63/583,228, filed on Sep. 15, 2023.

(51) Int. Cl. C12N 15/113; A61K 38/37; A61K 38/48; A61K 45/06; A61P 7/04

(52) U.S. Cl. CPC C12N 15/113; A61K 38/37; A61K 38/4833; A61K 38/4846; A61K 45/06; A61P 7/04; C12Y 304/21005; C12Y 304/21006; C12Y 304/21021; C12Y 304/21022; C12N 2310/14; C12N 2310/351; C12N 2320/32; C12N 2320/35

(57) ABSTRACT

The present disclosure provides methods for using fitusiran to treat hemophilia A in patients who have previously been treated with emicizumab.

### CROSS-REFERENCE TO RELATED APPLICATION

This application claims priority from U.S. Patent Application No. 63/583,228, filed Sep. 15, 2023, the disclosure of which is incorporated herein by reference in its entirety.

### SEQUENCE LISTING

The instant application contains a Sequence Listing which has been submitted electronically in XML format and is hereby incorporated by reference in its entirety. Said XML copy, created on Sep. 12, 2024, is named 122548_WO022_SL.xml and is 26,873 bytes in size.

### BACKGROUND OF THE INVENTION

Hemophilia A and hemophilia B are X-linked recessive inherited bleeding disorders, characterized by deficiency of coagulation Factor VIII (FVIII) or Factor IX (FIX), leading to a profound defect of thrombin generation with impaired hemostasis and increased risk of bleeding. Hemophilia A is found in approximately 1 in 4,000 males whereas hemophilia B is five times less common and seen in approximately 1 in 20,000 males. The disease phenotype presents similarly in hemophilia A and B.

Hemophilia is classified as mild (factor levels 6% to 30%), moderate (factor levels 1% to 5%), or severe (factor levels <1%) based on clotting factor activity relative to normal (healthy, non-hemophiliac plasma levels of factor are 50% to 150%). Patients with mild hemophilia typically experience bleeding after a serious injury or surgery. Patients with moderate hemophilia experience bleeding episodes associated with injuries, and may have spontaneous bleeding episodes. Severe hemophilia patients experience substantial bleeding with injury and may have frequent spontaneous bleeding episodes resulting in debilitating musculoskeletal damage that can markedly impair a patient's mobility and quality of life (QOL).

The hemostatic system aims to maintain the integrity of the vasculature by protecting against bleeding from vessel lesions combined with multiple options to prevent thrombosis. This hemostatic balance is achieved through an orchestrated regulation of both procoagulant (e.g., Factor V (FV), Factor VII (FVII), FVIII, FIX, Factor X (FX)) and anticoagulant (e.g., antithrombin, protein C/protein S and tissue factor pathway inhibitor) factors. Recent studies have suggested that coinheritance of a deficiency in natural anticoagulants may contribute to a milder phenotype in patients with hemophilia.

Antithrombin (AT) is a liver-expressed natural anticoagulant that plays a key role in inhibiting thrombin. AT acts as an inhibitor of FVIIa and FXa, which are typically at normal levels in patients with hemophilia A or B. Extensive preclinical in vitro and in vivo studies have described reduction of AT as a potential safe and effective way to correct thrombin generation in both hemophilia A and B and control against microvascular and macrovascular traumatic bleeding episodes. Therefore, suppression of AT production is being investigated as a potential hemophilia treatment.

Prophylaxis is the recommended standard of care for people with severe bleeding phenotype in people with hemophilia A. While clotting factor concentrates replacement therapy has well established safety and efficacy profiles, it is associated with high treatment burden due to requirement of IV administration on a frequent schedule (two to three times per week or more) to prophylactically maintain hemostasis. Bypassing agent (BPA) prophylaxis has a higher treatment burden with infusion frequency that can be as high as daily but may not be feasible for patients with inhibitors because of cost, increased infusion frequency (e.g. recombinant activated factor VII [rFVIIa]), large infusion volumes (aPCC), theoretical risk for thrombosis, and incomplete correction of the hemostatic defect that results in more breakthrough bleeding.

Emicizumab, a bispecific antibody that mimics the function of FVIIIa, is approved for people with hemophilia A irrespective of age and inhibitor status. While emicizumab has shown promise in this population, there have been observed clinical events of thrombotic microangiopathy and thrombosis especially when used in combination with aPCC that requires risk mitigation strategies.

There remains a need for alternative treatments for patients having hemophilia, to prevent or reduce the frequency of bleeding episodes, while reducing treatment burden, improving clinical outcomes, and enhancing quality of life.

### SUMMARY OF THE INVENTION

The present disclosure provides a method of routine prophylaxis to prevent or reduce the frequency of bleeding episodes in a human patient having hemophilia A who has been on prophylactic treatment with emicizumab, comprising subcutaneously administering to the human patient in need thereof a first dose of fitusiran between about one and about six months after termination of the prophylactical treatment with emicizumab, optionally wherein the first dose of fitusiran is in the amount of about 10 or about 20 mg, and subsequent doses of fitusiran in the amount of about 10-80 mg about once a month (QM) or about every four weeks (Q4W), or about once every other month (Q2M) or about once every eight weeks (Q8W). The disclosure also provides a method of maintaining an appropriate thrombin generation (AG) potential in a human patient having hemophilia A with or without inhibitors who has been on prophylactic treatment with emicizumab, comprising subcutaneously administering to the human patient in need thereof a first dose of fitusiran between about one and about six months after termination of the prophylactical treatment with emicizumab, optionally wherein the first dose of fitusiran is in the amount of about 10 or about 20 mg, and subsequent doses of fitusiran in the amount of about 10-80 mg about once a month (QM) or about every four weeks (Q4W), or about once every other month (Q2M) or about once every eight weeks (Q8W).

In some embodiments, the patient is a hemophilia A patient with inhibitors. In some embodiments, the patient is a hemophilia A patient without inhibitors.

The patient may have been on prophylactic treatment with emicizumab at a dose of about 1.5 mg/kg to about 6 mg/kg about once a week (QW) to about Q4W, or about QM. In some embodiments, the patient has been on prophylactic treatment with emicizumab at a dose of about 1.5 mg/kg about QW, or about 3 mg/kg about once every other week (Q2W). In some embodiments, the patient has been on prophylactic treatment with emicizumab at a dose of about 6 mg/kg about Q4W or about QM.

In some embodiments, the prophylactic emicizumab treatment in the patient is terminated about two months before the first dose of fitusiran. In some embodiments, the prophylactic emicizumab treatment in the patient is terminated about two and a half months before the first dose of fitusiran.

In one aspect, the patient is an adolescent or adult patient, and the first dose and second dose of fitusiran are each about 20 mg and given about two months or about eight weeks apart. In another aspect, the patient is a pediatric patient, and the first dose and second dose of fitusiran are each about 10 mg and given about two months or about eight weeks apart.

In some embodiments, the method further comprises obtaining a measurement of an antithrombin (AT) level after two doses of fitusiran or at a steady state (SS) in the patient; and performing one of the following steps: (i) if the AT level is 15-35%, repeating administration of fitusiran at the amount of the first dose about Q2M or about every eight weeks (Q8W); (ii) if the AT level is >35%, subcutaneously administering to the patient fitusiran at about 50 mg about Q2M or Q8W, or (iii) if the AT level is <15%, discontinuing or pausing fitusiran treatment.

In some embodiments, the method comprises after step (ii), (a) subcutaneously administering to the patient fitusiran at about 50 mg about QM or about Q4W if the patient has two SS measurements of AT level that are >35%, and then (a1) subcutaneously administering to the patient fitusiran at 80 mg about QM or about Q4W if the patient has two SS measurements of AT level that are >35%, and de-escalating to the preceding dosing regimen if the patient has more than one measurement of AT level that is <15%; or (a2) de-escalating to the dosing regimen of (ii) if the patient has more than one measurement of AT level that is <15%; (b) subcutaneously administering to the patient fitusiran about 20 mg if the patient has more than one measurement of AT level that is <15%, and then (b1) repeating administration of fitusiran at about 20 mg QM when the patient has a measurement of AT level that is ≥22%; or (b2) discontinuing fitusiran treatment if the patient has more than one measurement of AT level that is <15%.

In some embodiments, the method further comprises administering an effective amount of a replacement factor VIII or bypassing agent (BPA) to the patient to treat a bleeding episode that occurs seven or fewer days after the first fitusiran dose, wherein the effective amount of the replacement factor or BPA is administered according to the patient's prior dosing regimen of replacement factor or BPA.

The method may also comprise administering an effective amount of replacement factor VIII or bypassing agent (BPA) to the patient to treat a bleeding episode that occurs eight or more days after the first fitusiran dose, wherein the effective amount of the replacement factor or BPA is reduced as compared to the recommended effective amount of the replacement factor or BPA for patients who are not on fitusiran therapy. In some embodiments, the patient is a patient without factor VIII inhibitors, and the replacement factor is factor VIII and a single dose of factor VIII is no more than 20 IU/kg and optionally is 10 IU/kg, optionally wherein the factor VIII administration is repeated, if needed, in no less than 24 hours. In another aspect, the patient is a patient with factor VIII inhibitors, and the BPA is activated prothrombin complex concentrate (aPCC) and a single dose of aPCC is no more than 50 U/kg and optionally is 30 U/kg, optionally wherein the aPCC administration is repeated, if needed, in no less than 24 hours. In some embodiments, aPCC is administered to treat a bleeding episode no sooner than six months after the last dose of emicizumab or no sooner than four months after the first dose of fitusiran. In some embodiments, the patient is a patient with factor VIII inhibitors, and the BPA is recombinant factor VIIa (rFVIIa) and a single dose of rFVIIa is no more than 45 μg/kg, optionally wherein the rFVIIa administration is repeated, if needed, in no less than two hours.

In another aspect, the present disclosure provides fitusiran for use in a method of the present disclosure. The present disclosure also provides use of fitusiran in the manufacture of a medicament for use in a method of the present disclosure.

In one aspect, the present disclosure provides fitusiran for use in routine prophylaxis to prevent or reduce the frequency of bleeding episodes characterized in that the human patient having hemophilia A has been on prophylactic treatment with emicizumab and comprising subcutaneously administering to the human patient in need thereof a first dose of fitusiran between one and six months after termination of the prophylactical treatment with emicizumab, optionally wherein the first dose of fitusiran is in the amount of 10 or 20 mg, and subsequent doses of fitusiran in the amount of 10-80 mg once a month (QM) or every four weeks (Q4W), or once every other month (Q2M) or once every eight weeks (Q8W).

In another aspect, the present disclosure provides fitusiran for use in treatment of hemophilia A characterized in that the treatment comprises routine prophylaxis to prevent or reduce the frequency of bleeding episodes, the human patient having hemophilia A has been on prophylactic treatment with emicizumab and comprising subcutaneously administering to the human patient in need thereof a first dose of fitusiran between one and six months after termination of the prophylactical treatment with emicizumab, optionally wherein the first dose of fitusiran is in the amount of 10 or 20 mg, and subsequent doses of fitusiran in the amount of 10-80 mg once a month (QM) or every four weeks (Q4W), or once every other month (Q2M) or once every eight weeks (Q8W).

In another aspect, the present disclosure provides fitusiran for use in the treatment of hemophilia A with or without inhibitors characterized in that the treatment comprises maintaining an appropriate thrombin generation (AG) potential, the human patient having hemophilia A has been on prophylactic treatment with emicizumab and comprising subcutaneously administering to the human patient in need thereof a first dose of fitusiran between one and six months after termination of the prophylactical treatment with emicizumab, optionally wherein the first dose of fitusiran is in the amount of 10 or 20 mg, and subsequent doses of fitusiran in the amount of 10-80 mg once a month (QM) or every four weeks (Q4W), or once every other month (Q2M) or once every eight weeks (Q8W).

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 shows the expanded structural formula, chemical formula, and molecular mass of fitusiran sodium.

FIG. 2 shows a fitusiran dose regimen. SS: steady state.

FIG. 3 is a flow chart depicting a fitusiran escalation/de-escalation scheme for pediatric patients with a body weight of 22 kg to <45 kg. Participants will start fitusiran at a dose of 10 mg every 4 weeks. AT: antithrombin activity; QM=once monthly; SS=steady state. *: within a 12-month period; **: start of dosing after de-escalation from higher dose to occur only after centrally measured AT activity levels ≥22%.

FIG. 4 is a flow chart depicting a fitusiran escalation/de-escalation scheme for pediatric patients with a body weight of 8 kg to <22 kg. Participants will start fitusiran at a dose of 5 mg every 4 weeks. Abbreviations and symbols are the same as explained for FIG. 3 above.

FIG. 5 shows a clinical trial study design for patients who have been previously treated prophylactically with emicizumab before beginning fitusiran treatment. AT: antithrombin; D: day; EOT: end of treatment; M: month; QM: every month; Q2W: every two weeks; QW: every week.

FIG. 6 is a set of line graphs indicating the predicted percentage of FVIII-like activity in patients following emicizumab cessation (Panel A) and following the start of fitusiran administration (Panel B). The additive emicizumab FVIII-like activity and fitusiran FVIII-like activity are shown in (Panel C).

### DETAILED DESCRIPTION OF THE INVENTION

The present disclosure provides methods for transitioning hemophilia A patients with or without inhibitors from emicizumab prophylaxis to fitusiran prophylaxis.

Emicizumab is a bispecific antibody that serves as a mimetic for activated FVIII. It has been approved in the United States for routine prophylaxis to prevent or reduce the frequency of bleeding episodes in adult and pediatric patients ages newborn and older with hemophilia A (congenital factor VIII deficiency) with or without factor VIII inhibitors. There have been reported difficulties associated with emicizumab treatment. In some patients, especially patients treated with emicizumab in combination with the bypassing agent (BPA) activated prothrombin complex concentrates (aPCC), thrombotic microangiopathy and thrombosis have been observed, necessitating risk mitigation strategies and a black box warning on the U.S. label. There is also a risk that emicizumab may induce development of neutralizing drug antibodies. In addition, dosing with emicizumab every other week or monthly may require multiple injections due to high injection volume, increasing pain at injection sites (see, e.g., Kruis et al., Haemophilia (2023) 29 (2): 689-91). Furthermore, approximately 30-40% of patients treated with emicizumab are reported to continue to experience spontaneous bleeding episodes (see, e.g., Callaghan et al., Blood. (2001) 137 (16): 2231-42).

Emicizumab is known to interfere with clotting-based assays, an effect that can last for up to six months following the last dose of emicizumab, confounding analytical results for hemophilia patients attempting to transition to standard of care (see, e.g., Pasi et al., N Engl J Med. (2017) 377 (9): 819-28). This six-month period has been adopted as a waiting time to switch to any other hemostatic therapy in case of emicizumab failure, but as emicizumab levels lower, the risk of spontaneous bleeding episodes increases. There are currently no data to describe how patients can switch from emicizumab to other non-factor treatment options earlier than a full six-month washout period following the last dose of emicizumab. In the absence of guidance on how to safely switch from emicizumab to another therapy earlier than six months, there will be limitations in clinical practice, leading to delay in switching to potential optimal prophylaxis.

The present disclosure provides methods for switching patients from emicizumab prophylaxis to fitusiran prophylaxis without having to wait out the six-month washout period. Fitusiran is a N-acetyl-galactosamine (GalNAc) small interfering ribonucleic acid (siRNA) conjugate that reduces production of antithrombin (AT), leading to lower plasma AT activity levels. By reducing plasma AT, fitusiran is designed to improve thrombin generation and hemostasis in individuals with hemophilia, regardless of hemophilia type or presence of inhibitory antibodies to FVIII or FIX. Fitusiran may be administered less frequently than emicizumab.

The present methods introduce fitusiran prophylaxis to hemophilia A patients earlier than the six-month waiting period following the final dose of emicizumab, allowing patients who are not effectively treated by emicizumab to switch treatment methods sooner, without having to rely on the more burdensome factor or BPA therapies for prolonged periods of time. The present methods can maintain a favorable benefit-risk balance for such patients. In some embodiments, the patient is an adult patient 18 years of age or older. In some embodiments, the patient is an adolescent patient who is from 12 to less than 18 years of age. In some embodiments, the patient is a pediatric patient who is less than 12 years of age (e.g., a patient who is from one to less than twelve years of age).

### I. Fitusiran Pharmaceutical Compositions

Hemophilia results in a profound defect in thrombin generation, and further, hemophilia severity is correlated with the inability to generate thrombin. Without being bound by theory, it is believed that fitusiran-mediated lowering of antithrombin (AT) levels will increase thrombin generation and thus improve hemostasis in patients with hemophilia. Antithrombin is encoded by the SERPINC1 gene.

Fitusiran, whose structure is described herein, is a synthetic, chemically modified double-stranded small interfering RNA (siRNA) oligonucleotide covalently linked to a tri-antennary N-acetyl-galactosamine (GalNAc) ligand targeting the AT3 mRNA in the liver, thereby suppressing the synthesis of antithrombin. The nucleosides in each strand of fitusiran are connected through either 3′-5′ phosphodiester or phosphorothioate linkages, thus forming the sugar-phosphate backbone of the oligonucleotide.

The sense strand and the antisense strand of fitusiran contain 21 and 23 nucleotides, respectively. The 3′-end of the sense strand is conjugated to the GalNAc containing moiety (referred to as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, and WO 2019/014187.

The two nucleotide strands of fitusiran are shown below:

sense strand:
(SEQ ID NO: 1)
5′Gf-ps-Gm-ps-Uf-Um-Af-Am-Cf-Am-Cf-Cf-Af-Um-Uf-
Um-Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-Gm-Gm-
Uf-Gm-Uf-Um-Af-Am-Cf-Cm-ps-Am-ps-Gm 3′,

wherein
Af=2′-fluoroadenosine (i.e., 2′-deoxy-2′-fluoroadenosine)
Cf=2′-fluorocytidine (i.e., 2′-deoxy-2′-fluorocytidine)
Gf=2′-fluoroguanosine (i.e., 2′-deoxy-2′-fluoroguanosine)
Uf=2′-fluorouridine (i.e., 2′-deoxy-2′-fluorouridine)
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt and wherein L96 has the following formula:

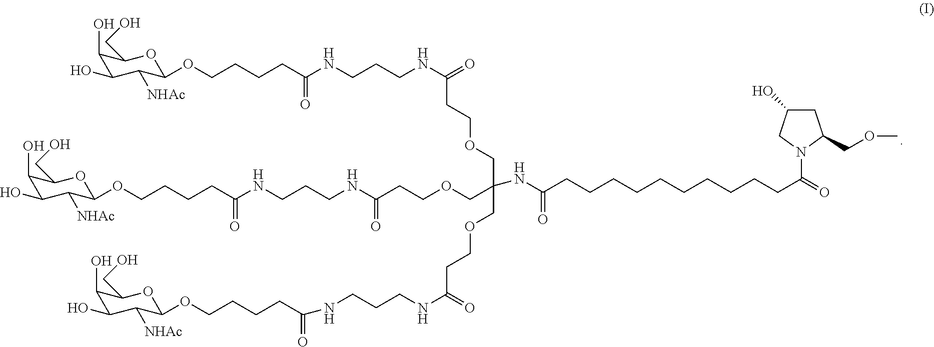 [SMILES: COC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

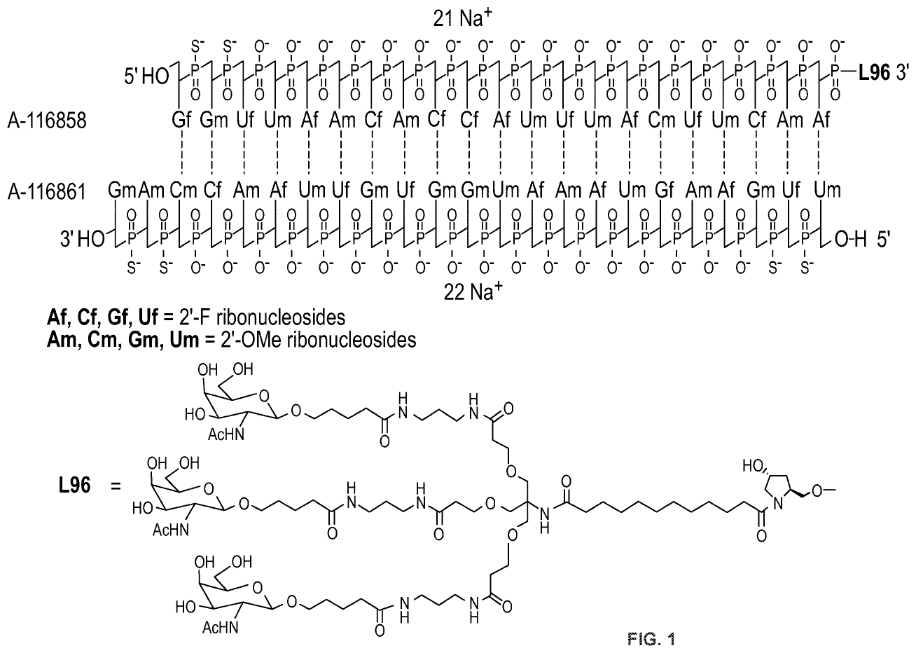

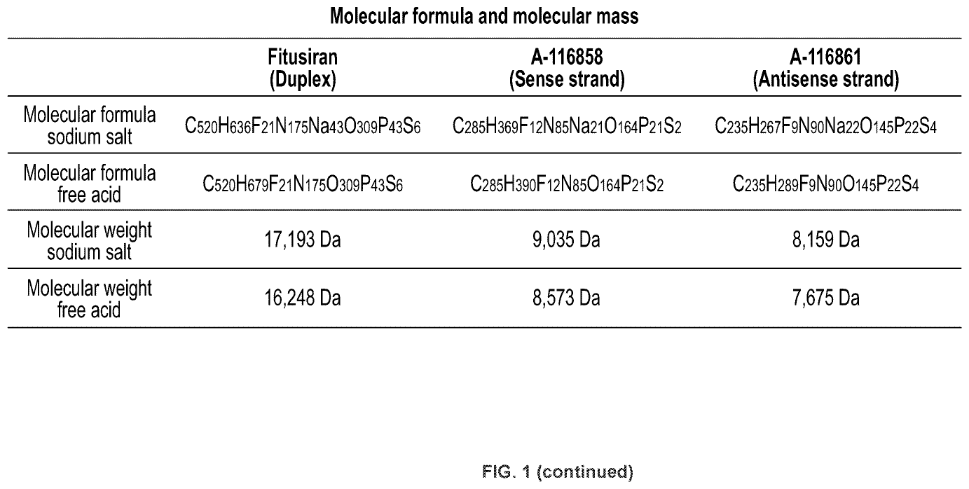

The expanded structural formula, molecular formula, and molecular weight of fitusiran (sodium form) are shown in FIG. 1. As used herein, the term 2′-fluoroadenosine is used interchangeably with the term 2′-deoxy-2′-fluoroadenosine; the term 2′-fluorocytidine is used interchangeably with the term 2′-deoxy-2′-fluorocytidine; the term 2′-fluoroguanosine is used interchangeably with the term 2′-deoxy-2′-fluoroguanosine, and the term 2′-fluorouridine is used interchangeably with the term 2′-deoxy-2′-fluorouridine.

The structure of fitusiran can also be described using the following diagram, wherein the X is O:

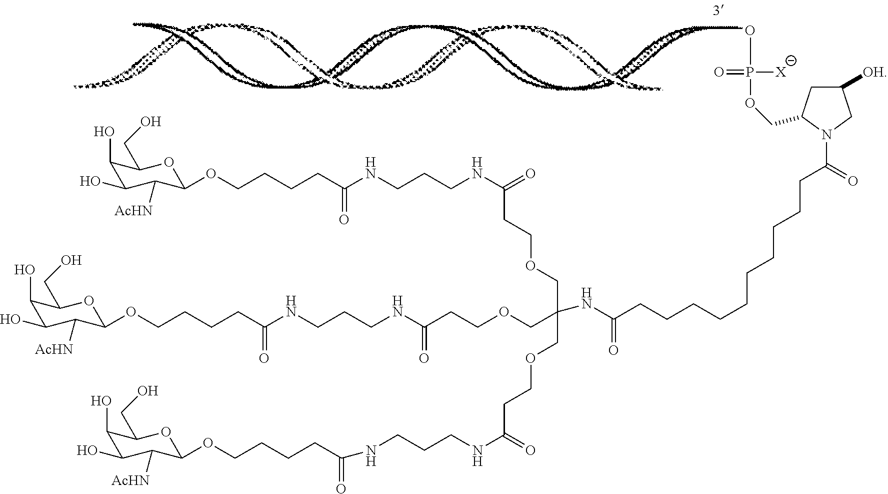

For use in the present treatment methods, fitusiran may be provided in a pharmaceutical composition comprising it and a pharmaceutically acceptable excipient. In certain embodiments, fitusiran is in sodium salt form.

In some embodiments, fitusiran is provided in an aqueous solution at a concentration of about 1 to about 200 mg/mL (e.g., about 50 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical composition comprises fitusiran at a concentration of about 6.25, about 12.5, about 25, about 50, about 75, about 100, about 125, about 150, or about 200 mg/mL.

Unless otherwise indicated, a fitusiran weight recited in the present disclosure is the weight of fitusiran free acid (the active moiety), even though fitusiran is injected to patients subcutaneously in its sodium form (in an aqueous solution). For example, 100 mg/mL fitusiran means 100 mg of fitusiran free acid (equivalent to 106 mg fitusiran sodium, the drug substance) per mL.

In some embodiments, the pharmaceutical compositions comprise fitusiran in a phosphate-buffered saline. The phosphate concentration in the solution may be about 1 to 10 mM (e.g., 2, 3, 4, 5, 6, 7, 8, or 9 mM), with a pH of 6.0-8.0. The pharmaceutical compositions herein may include a preservative such as EDTA. Alternatively, the pharmaceutical compositions are preservative-free. In particular embodiments, the fitusiran pharmaceutical composition is preservative-free and comprises, consists of, or consists essentially of about 100 mg of fitusiran per mL of a 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the composition to 7.0.

The pharmaceutical composition may be provided in a container (e.g., a vial or a syringe). The container may contain single or multiple doses. In some embodiments, the container is a single-use container (e.g., a single-use ampule or a single-use syringe such as a single-use pre-filled syringe), with each container containing about 10 to about 100 mg fitusiran (e.g., about 10 mg, about 20 mg, about 25 mg, about 40 mg, about 50 mg, or about 80 mg). The fitusiran may be provided in a solid form in the container and reconstituted in an aqueous solution (e.g., PBS) prior to use, with the reconstituted solution containing about 1 to about 150 mg/mL (e.g., 100 mg/mL) fitusiran. In some embodiments, fitusiran is provided in sodium salt form in a single-use glass vial or a single-use prefilled syringe (e.g., one with a safety system). In further embodiments, each vial or syringe contains about 80 mg of fitusiran in about 0.8 mL (or about 50 mg of fitusiran in about 0.5 mL, about 20 mg of fitusiran in about 0.2 mL, or about 10 mg of fitusiran in about 0.1 mL) of 5 mM phosphate buffered saline solution (pH 7.0); and the solution is administered to patients through subcutaneous injection. The solution can be stored at 2 to 30° C. (e.g., 2 to 8° C.).

In particular embodiments, the fitusiran composition for subcutaneous injection contains fitusiran in a 5 mM phosphate buffered saline having 0.64 mM NaH₂PO₄, 4.36 mM Na₂HPO₄, and 84 mM NaCl at pH 7.0. In certain embodiments, the composition of fitusiran solution for subcutaneous injection is shown in Table 1:

TABLE 1
Exemplary Formulation

| | Composition | | | |
|---|---|---|---|---|
| | | | Per unit | Per unit |
| | Per- | | (2 mL | (1 mL |
| | centage | Per ml | vial) | syringe) |
| Components | [%] | [mg] | [mg] | [mg] |
| Fitusiran (active moiety) | 10 | 100 [106] | 80 [84.8] | 80 [84.8] |
| [equivalent to | | | | |
| fitusiran sodium] | | | | |
| Sodium chloride | 0.49 | 4.909 | 3.927 | 3.927 |
| Dibasic sodium | 0.12 | 1.169 | 0.935 | 0.935 |
| phosphate (heptahydrate) | | | | |
| Monobasic sodium | <0.01 | 0.0885 | 0.0708 | 0.0708 |
| phosphate (monohydrate) | | | | |
| Phosphoric acid, | — | q.s. | q.s. | q.s. |
| concentrated | | pH 7.0 | pH 7.0 | pH 7.0 |
| Sodium hydroxide | — | q.s. | q.s. | q.s. |
| | | pH 7.0 | pH 7.0 | pH 7.0 |
| Water for injection | q.s. | q.s. | q.s. | q.s. |
| | 100 | 1 mL | 0.8 mL | 0.8 mL |

q.s.: quantum satis.

In some embodiments, fitusiran is packaged in a pre-filled pen containing 50 mg of fitusiran (equivalent to 53 mg fitusiran sodium). In some embodiments, each single-use pre-filled pen contains 50 mg of fitusiran in 0.5 mL solution at a concentration of 100 mg/mL. In some embodiments, each pre-filled pen is designed to deliver 50 mg fitusiran in 0.5 mL which also contains: sodium chloride (2.455 mg), dibasic sodium phosphate (0.585 mg), monobasic sodium phosphate (0.044 mg), phosphoric acid (q.s. pH 7.0), sodium hydroxide (q.s. pH 7.0), and water for injection (q.s. 0.5 mL). Fitusiran may be supplied as a sterile, clear solution for subcutaneous injection in the 50 mg PFP. In some embodiments, the PFP contains a single dose of fitusiran and comprises a prefilled syringe (PFS) assembled with an automated injection system (pen). The PFS is a 1 mL colorless type 1 glass syringe with a 29 gauge, (½ inch) stainless steel staked needle covered with a rigid needle shield. The syringe is closed with a rubber coated (bromobutyl gray) plunger stopper.

In some embodiments, fitusiran is packaged in a vial containing 20 mg of fitusiran (equivalent to 21 mg fitusiran sodium). In these embodiments, tach single-use vial contains 20 mg of fitusiran in 0.2 mL solution at a concentration of 100 mg/mL. In some embodiments, each vial contains 20 mg fitusiran in 0.2 mL which also contains: sodium chloride (0.982 mg), dibasic sodium phosphate (0.234 mg), monobasic sodium phosphate (0.018 mg), phosphoric acid (q.s. pH 7), sodium hydroxide (q.s. pH 7.0), and water for injection (q.s. 0.2 mL). Fitusiran may be supplied as a sterile, clear solution for subcutaneous injection in the vial. The vial comprises a single dose of fitusiran in a 2 mL colorless Type-1 glass vial with a bromobutyl rubber stopper and an aluminum polypropylene colored crimping cap.

In certain embodiments, the composition of fitusiran solution for subcutaneous injection is shown in Table 1B below.

TABLE 1B
Fitusiran Formulations

| Components | Formulation 1 (mg) | Formulation 2 (mg) |
|---|---|---|
| Fitusiran [Na salt] | 12.5 [13.25] | 100 [106] |
| NaH₂PO₄*H₂O | 0.388 | 0.0885 |
| Na₂HPO₄*7H₂O | 0.586 | 1.169 |
| NaCl | 8.7 | 4.909 |
| 0.1N NaOH | q.s. | q.s. |
| 0.1M H₃PO₄ | q.s. | q.s. |
| Purified water | Ad 1 mL | Ad 1 mL |

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH).

### II. Therapeutic Use of Fitusiran

Fitusiran can suppress liver production of antithrombin (AT). In its role as an anti-coagulant, AT regulates hemostasis by directly targeting thrombin production or by inactivating uncomplexed FXa, which in turn reduces thrombin production (Quinsey et al., Int J Biochem Cell Biol. (2004) 36 (3): 386-9). Fitusiran may be used to treat those who have impaired hemostasis. For example, fitusiran can be used to treat patients with hemophilia A with or without inhibitors for routine prophylaxis to prevent or reduce the frequency of bleeding episodes. In particular embodiments, fitusiran is used to treat adult and adolescent patients (at least twelve years of age) with hemophilia A (congenital factor VIII deficiency) with or without inhibitors. The present methods include administering to the hemophilia patient (e.g., a hemophilia A patient) in need thereof a therapeutically effective amount of fitusiran. “Therapeutically effective amount” refers to the amount of fitusiran that helps the patient to achieve a desired clinical endpoint.

In certain embodiments, the pharmaceutical composition is administered by subcutaneous injection at a dose strength of, for example, about 10 to about 100 mg (e.g., about 10 to about 95 mg, about 40 to about 90 mg, about 50 to about 100 mg, about 50 to about 90 mg, about 50 to about 85 mg, or about 50 to about 80 mg) per dose. In particular embodiments, fitusiran is administered subcutaneously at about 10, about 20, about 50, or about 80 mg (weight of active moiety) per dose in a PBS solution as described above.

A plurality of fitusiran doses may be administered to a patient at an interval of about one, about two, about three, about four, about five, about six, about seven, or about eight weeks, or of about one, about two, or about three months. In particular embodiments, a fixed dose of fitusiran (e.g., about 50 or about 80 mg subcutaneous injection) is administered to a hemophilia patient (e.g., a hemophilia A or hemophilia B patient who is twelve years or older and who has or has not developed inhibitors) about once every four weeks or about once a month or about once every eight weeks or about once every other month.

In some embodiments, the present methods involve measurement of AT levels in a patient. AT measurements can be performed by well-established methods, including both kinetic and chromogenic assays. One commonly used method is the INNOVANCE™ Antithrombin assay (Siemens Healthineers, Malvern, PA; U.S. FDA 510(k) #K081769). INNOVANCE™ is a chromogenic assay that quantifies functionally active AT in human citrated plasma based on the inhibition of an excess of factor Xa by AT. The assay may be performed by using an automated coagulation instrument (e.g., Siemens BCS® XP, Sysmex® CA-600 and CS Systems, or Atellica® COAG 360 System), and may be calibrated using Siemens standard human plasma (SHP) with a defined value of AT activity calibrated against World Health Organization (WHO) reference plasma. An equivalent assay is the Dade Behring Berichrom™ Antithrombin III assay (Dade Behring Marburg GmbH, Marburg, Germany; U.S. FDA 510(k) #K933125). Each measurement may be controlled by two independent controls (low and normal value) also calibrated against the WHO standard. The AT activity (%) in a plasma sample is calculated against the WHO reference plasma. 100% AT level is defined as 1 unit of antithrombin activity in 1 mL of reference plasma sample. The limit of detection of the INNOVANCE™ assay is 6.0% based on the assay's U.S. FDA 510(k) decision summary. AT levels range from about 80% to about 120% in the general population.

It has been observed that the risk of arterial thrombotic events among patients receiving fitusiran may increase with AT levels <10%. To mitigate the risk of vascular thrombotic events while maintaining a favorable benefit-risk balance for patients on fitusiran, a patient who has been on prophylactic treatment with emicizumab, for example an adult patient (≥18 years of age) or an adolescent patient (12 to 17 years of age, inclusive) who has been on prophylactic treatment with emicizumab, may start on a fitusiran therapy by subcutaneous injection of about 20 mg fitusiran about every two months (or about every eight weeks). The patient's AT level is monitored periodically (e.g., about every one, two, three, four, five, six, seven, or eight weeks, or about every one, two, three, four, five, or six months). If the patient has two AT measures of <15% (e.g., <10%), the patient will discontinue fitusiran treatment. In some embodiments, upon the first AT level <15%, the patient has another AT activity level sample drawn within about a month (e.g., within about one or about two weeks). If this result is <15%, this will be considered the second AT <15%. Patients receiving fitusiran at a dose of about 20 mg about Q2M with more than one (e.g., two) AT activity levels <15% will discontinue fitusiran.

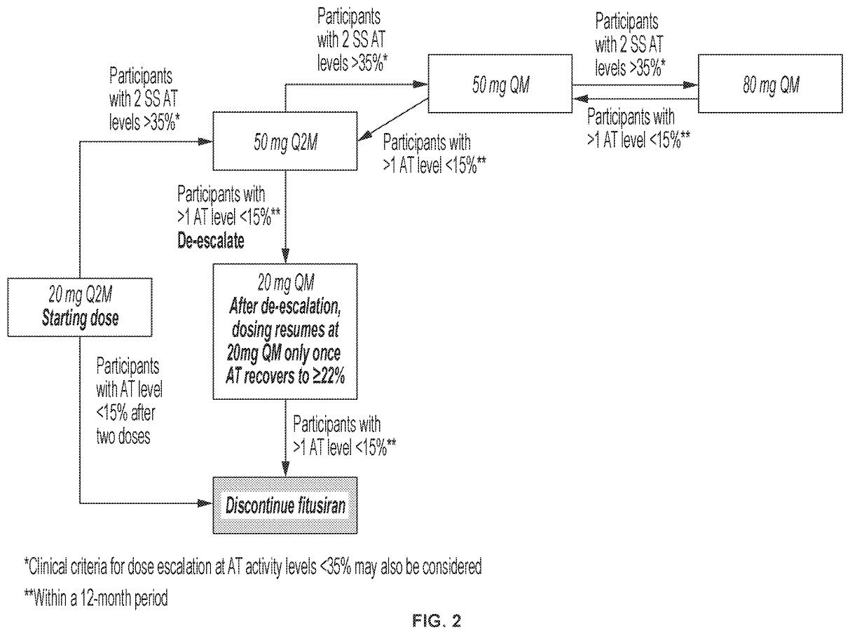

However, if the 20 mg/Q2M (or Q8W) adult or adolescent patient has two AT measures of >35%, the patient will escalate the dosing regimen as illustrated in FIG. 2. In the approach of FIG. 2, the patient may receive fitusiran at about 50 mg about every two months (or about every eight weeks); if the patient has two AT measurements of >35% under the 50 mg/Q2M (Q8W) regimen, the patient may receive fitusiran at about 50 mg about every month (or about every four weeks); if the patient has two AT measurements of >35% under the 50 mg/QM (Q4W) regimen, the patient may receive fitusiran at about 80 mg about every month (or about every four weeks)

Patients may de-escalate fitusiran dosing as shown in FIG. 2. For example, patients who have discontinued fitusiran after having more than one (e.g., 2) AT activity levels <15% when receiving fitusiran at a dose of 50 mg Q2M may receive fitusiran at a dose of 20 mg Q2M once their AT levels have returned to ≥22%; patients who have discontinued fitusiran after having more than one (e.g., 2) AT activity levels <15% when receiving fitusiran at a dose of about 50 mg about QM (or about Q4W) may receive fitusiran at a dose of 50 mg Q2M once their AT levels have returned to ≥22%; and patients who have discontinued fitusiran after having more than one (e.g., 2) AT activity levels <15% when receiving fitusiran at a dose of 80 mg QM may receive fitusiran at a dose of about 50 mg about QM (or about Q4W) once their AT levels have returned to ≥22%. Patients receiving fitusiran at a dose of 20 mg QM (or Q4W) with more than one (e.g., two) AT activity level <15% will discontinue fitusiran treatment.

In the above dose finding regimens, AT measurements for dosing determination are those taken during steady state (SS) of AT activity, i.e., once the patient's AT levels have been stabilized (at low AT activity range) after fitusiran treatment. The SS is typically reached after two or three doses of fitusiran. AT measurements for dosing determination are taken at an appropriate interval (e.g., about every four weeks or about every eight weeks). In the above dose finding regimens, the starting dose of 20 mg fitusiran Q2M is included as an illustrative example. For example, a starting dose of fitusiran may be 50 mg Q2M, 20 mg Q2M, 20 mg QM, or 10 mg QM. Dose escalation and de-escalation can then be carried out accordingly from each starting dose. For example, a starting dose of 50 mg Q2M fitusiran can be escalated to, 50 mg QM or 80 mg QM, optionally sequentially in that order, or de-escalated to 20 mg QM or 20 mg Q2M.

Pediatric Patients with a Higher Body Weight

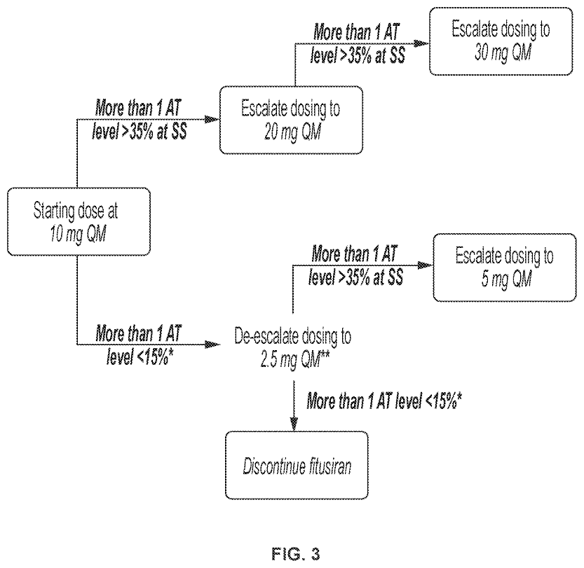

Patients with a body weight of 22 kg to <45 kg who have been on prophylactic treatment with emicizumab may begin with a starting dose of 10 mg fitusiran every month (or every four weeks). The patient's AT level may be monitored periodically (e.g., every one, two, three, four, five, six, seven, or eight weeks, or every one, two, three, four, five, or six months). An exemplary escalation and de-escalation scheme for pediatric patients is illustrated in FIG. 3.

In some embodiments, patients treated with a starting dose of 10 mg fitusiran every month (or every four weeks) de-escalate their fitusiran dose regimen. In some embodiments, upon the first AT level <15%, the patient has another AT activity level sample drawn within a month (e.g., within one or two weeks). If this result is <15%, this is considered the second AT <15%. Patients receiving fitusiran at a dose of 10 mg QM with more than 1 (e.g., 2) AT activity levels <15% will de-escalate to a dose of 2.5 mg fitusiran every month (or every four weeks). The patient may initiate treatment with the lower dose of fitusiran after their AT levels have returned to above 15%, e.g., ≥22%.

After de-escalating to a dose of 2.5 mg fitusiran every month, the patient's AT level may be again monitored periodically (e.g., every one, two, three, four, five, six, seven, or eight weeks, or every one, two, three, four, five, or six months). In some embodiments, upon the first AT level <15%, the patient has another AT activity level sample drawn within a month (e.g., within one or two weeks). If this result is <15%, this is considered the second AT <15%. Patients receiving fitusiran at a dose of 2.5 mg QM with more than 1 (e.g., 2) AT activity levels <15% may discontinue or pause fitusiran treatment.

Alternatively, after de-escalating to a dose of 2.5 mg fitusiran every month, the patient's AT level is again monitored periodically (e.g., every one, two, three, four, five, six, seven, or eight weeks, or every one, two, three, four, five, or six months). In some embodiments, upon the first steady state AT level >35%, the patient has another AT activity level sample drawn within a month (e.g., within one or two weeks). If this result is >35%, this is considered the second steady state AT >35%. Patients receiving fitusiran at a dose of 2.5 mg QM with more than 1 (e.g., 2) steady state AT activity levels >35% may escalate to a dose of 5 mg fitusiran once a month (or every four weeks).

In other embodiments, patients treated with a starting dose of 10 mg fitusiran every month (or every four weeks) may escalate their dose of fitusiran. The patient's AT level may be monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 10 mg QM with more than 1 (e.g., 2) steady state AT activity levels >35% may escalate to a dose of 20 mg fitusiran every month (or every four weeks).

After escalating to a dose of 20 mg fitusiran every month, the patient's AT level may be again monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 20 mg QM with more than 1 (e.g., 2) steady state AT activity levels >35% may escalate to a dose of 30 mg fitusiran once a month (or every four weeks).

In some embodiments, escalation is performed after AT activity levels at steady state remain >35% (see, e.g., FIG. 3).

Pediatric Patients with a Lower Body Weight

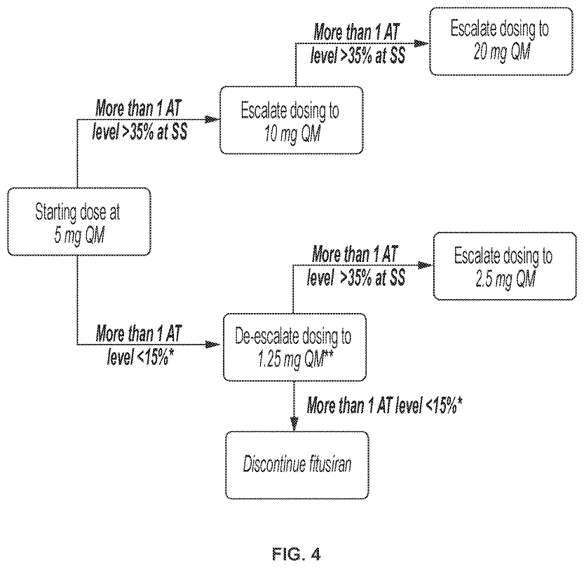

Patients with a body weight of 8 kg to <22 kg who have been on prophylactic treatment with emicizumab may begin with a starting dose of 5 mg fitusiran every month (or every four weeks). An escalation and de-escalation scheme for pediatric patients is illustrated in FIG. 4. The patient's AT level may be monitored periodically (e.g., every one, two, three, four, five, six, seven, or eight weeks, or every one, two, three, four, five, or six months).

In some embodiments, patients treated with a starting dose of 5 mg fitusiran every month (or every four weeks) may de-escalate their dose of fitusiran. In some embodiments, upon the first AT level <15%, the patient has another AT activity level sample drawn within a month (e.g., within one or two weeks). If this result is <15%, this is considered the second AT <15%. Patients receiving fitusiran at a dose of 5 mg QM with more than 1 (e.g., 2) AT activity levels <15% may de-escalate to a dose of 1.25 mg fitusiran every month (or every four weeks). The patient may initiate with the lower dose of fitusiran after their AT levels have returned to above 15%, e.g., ≥22%.

After de-escalating to a dose of 1.25 mg fitusiran every month, the patient's AT level may be again monitored periodically (e.g., every one, two, three, four, five, six, seven, or eight weeks, or every one, two, three, four, five, or six months). In some embodiments, upon the first AT level <15%, the patient has another AT activity level sample drawn within a month (e.g., within one or two weeks). If this result is <15%, this is considered the second AT <15%. Patients receiving fitusiran at a dose of 1.25 mg QM with more than 1 (e.g., 2) AT activity levels <15% may discontinue or pause fitusiran treatment.

Alternatively, after de-escalating to a dose of 1.25 mg fitusiran every month, the patient's AT level may again be monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 1.25 mg QM with more than 1 (e.g., 2) steady state AT activity levels >35% may escalate to a dose of 2.5 mg fitusiran once a month (or every four weeks).

In other embodiments, patients treated with a starting dose of 5 mg fitusiran every month (or every four weeks) may escalate their dose of fitusiran. The patient's AT level may be monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 5 mg QM with more than 1 (e.g., 2) steady state AT activity levels >35% may escalate to a dose of 10 mg fitusiran every month (or every four weeks).

After escalating to a dose of 10 mg fitusiran every month, the patient's AT level may again be monitored periodically (e.g., every one, two, three, four, five, six, seven, or eight weeks, or every one, two, three, four, five, or six months). In some embodiments, upon the first steady state AT level >35%, the patient has another steady state AT activity level sample drawn within a month (e.g., within one or two weeks). If this result is >35%, this is considered the second steady state AT >35%. Patients receiving fitusiran at a dose of 10 mg QM with more than 1 (e.g., 2) steady state AT activity levels >35% may escalate to a dose of 20 mg fitusiran once a month (or every four weeks).

In some embodiments, escalation is performed after AT activity levels at steady state remain >35% (see, e.g., FIG. 4).

In some embodiments, AT activity levels used for deciding whether to escalate a fitusiran dose amount or frequency are those measured at steady state (SS), i.e., once the patient's AT levels have been stabilized after fitusiran treatment. The SS is typically reached after two or three doses of fitusiran. AT measurements for dosing determination are taken at an appropriate interval (e.g., every four weeks or every eight weeks).

In the above pediatric dose finding regimens, the starting dose of 10 mg fitusiran QM or 5 mg fitusiran QM is discussed as an illustrative example. For example, a starting dose of fitusiran may be 20 mg QM, 10 mg Q2M, 7.5 mg Q2M, 5 mg Q2M, or 2.5 mg QM. Dose escalation and de-escalation can then be carried out accordingly from each starting dose. For example, a starting dose of 2.5 mg QM fitusiran can be escalated to 5 mg QM, 10 mg QM, 20 mg QM, 30 mg QM, or 50 mg QM, optionally sequentially in that order, or de-escalated to 2.5 mg Q2M or 1.25 QM.

An AT level of 10-35% (e.g., 10-25%, 15-35%, or 15-25%) is targeted to mitigate the risk of vascular thrombotic events while maintaining a favorable benefit-risk balance for patients on fitusiran. In some embodiments, the target AT level is 15-35%. Thus, so long as the patient reaches this targeted AT level, there is no need for the patient to receive a higher fitusiran dosage or more frequent dosing. That is, he remains on the current treatment regimen (i.e., maintenance regimen). For example, once the desired AT level is reached, the patient may be treated with a subcutaneous dose of fitusiran (e.g., 40-90 mg per dose) at an interval of, e.g., about every one, two, three, four, five, six, seven, or eight weeks, or about every one, two, three, four, five, or six months. In some embodiments, if the patient has two AT measurements of no greater than 35% while receiving fitusiran at a dose of 50 mg Q2M, he will maintain this dosing regimen, with no need to further escalate the dosage or dosing frequency. As another example, if the patient has two AT measurements of no greater than 35% while receiving 80 mg Q2M or 50 mg QM, he will remain on this dosing regimen, with no need to further escalate the dosage or dosing frequency (to, e.g., 80 mg QM). However, fitusiran treatment should be discontinued if a patient has more than one (e.g., two) AT measurements <15% (e.g., <10%) as a risk mitigation measure for vascular thrombotic events. Alternatively, the patient may resume treatment with a lower dose of fitusiran after their AT levels have returned to above 15%, e.g., ≥22%. In some embodiments of maintenance regimens, an adult or adolescent patient with hemophilia A with or without inhibitors is treated with a subcutaneous dose of fitusiran at about 50 mg per dose about every two months (or about every eight weeks). In other embodiments, an adult or adolescent patient with hemophilia A with or without inhibitors is treated with a subcutaneous dose of fitusiran at about 50 mg about every month (or about every four weeks). In yet other embodiments, an adult or adolescent patient with hemophilia A with or without inhibitors is treated with a subcutaneous dose of fitusiran at about 80 mg about every two months (or about every eight weeks). In yet other embodiments, an adult or adolescent patient with hemophilia A with or without inhibitors is treated with a subcutaneous dose of fitusiran at about 80 mg about every month (or about every four weeks). In yet other embodiments, an adult or adolescent patient with hemophilia A with or without inhibitors is treated with a subcutaneous dose of fitusiran at about 20 mg about every two months (or about every eight weeks). In yet other embodiments, an adult or adolescent patient with hemophilia A with or without inhibitors is treated with a subcutaneous dose of fitusiran at about 20 mg about every month (or about every four weeks). In yet other embodiments, an adult or adolescent patient with hemophilia A with or without inhibitors is treated with a subcutaneous dose of fitusiran at about 10 mg about every month (or about every four weeks).

A pediatric patient (i.e., a patient that is from 1 to less than 12 year(s) of age) with hemophilia A with or without inhibitors may be treated with a subcutaneous maintenance dose of fitusiran at about 1-50 mg per dose about every month (or about every four weeks). In some embodiments of maintenance regimens, a pediatric patient with hemophilia A with or without inhibitors is treated with a subcutaneous dose of fitusiran at about 50 mg per dose about every month (or about every four weeks). In some embodiments of maintenance regimens, a pediatric patient with hemophilia A with or without inhibitors is treated with a subcutaneous dose of fitusiran at about 30 mg per dose about every month (or about every four weeks). In other embodiments, a pediatric patient with hemophilia A with or without inhibitors is treated with a subcutaneous dose of fitusiran at about 20 mg about every month (or about every four weeks). In other embodiments, a pediatric patient with hemophilia A with or without inhibitors is treated with a subcutaneous dose of fitusiran about 10 mg about every month (or about every four weeks). In some embodiments of maintenance regimens, a pediatric patient with hemophilia A with or without inhibitors is treated with a subcutaneous dose of fitusiran at about 7.5 mg per dose about every month (or about every four weeks). In other embodiments, a pediatric patient with hemophilia A with or without inhibitors is treated with a subcutaneous dose of fitusiran at about 5 mg about every month (or about every four weeks). In other embodiments, a pediatric patient with hemophilia A with or without inhibitors is treated with a subcutaneous dose of fitusiran at about 2.5 mg about every month (or about every four weeks). In other embodiments, a pediatric patient with hemophilia A with or without inhibitors is treated with a subcutaneous dose of fitusiran at about 1.25 mg about every month (or about every four weeks). The AT monitoring process for pediatric patients may be that as described for adolescents and adults, with the desired AT level being also 15-35%. In some embodiments, the patient may start at a dose of 10 mg about Q2M or about Q8W, escalate to the stronger dosing regimen (higher dose and/or frequency) or de-escalate to the weaker dosing regimen (lower dose and/or frequency), or discontinuation, based on the AT levels, as described above for adolescents and adults.

In some embodiments, patients may receive periodic (e.g., about monthly or about every four weeks) AT monitoring for about twelve months following a change in fitusiran dosing regimen.

In some embodiments, once a patient stays on a maintenance regimen (e.g., 10 mg QM or Q4W, 20 mg Q2M or Q8W, 20 mg QM or Q4W, 50 mg Q2M or Q8W, 50 mg QM or Q4W, 80 mg QM or Q4W, or 80 mg Q2M or Q8W), the patient may receive less frequent AT monitoring. For example, his AT level may be monitored about every month, about every two months, about every three months, about every four months, about semi-annually, about annually, or about every two years.

The present methods include administering to the hemophilia A patient in need thereof a therapeutically effective amount of fitusiran. “Therapeutically effective amount” refers to the amount of fitusiran that helps the patient to achieve a desired clinical endpoint.

In some embodiments, a patient with hemophilia A with or without inhibitors is treated with a subcutaneous dose of fitusiran at about 50 mg per dose about every two months (or about every eight weeks). In other embodiments, a patient with hemophilia A with or without inhibitors is treated with a subcutaneous dose of fitusiran at about 50 mg about every month (or about every four weeks). In yet other embodiments, a patient with hemophilia A with or without inhibitors is treated with a subcutaneous dose of fitusiran at about 80 mg about every two months (or about every eight weeks). In yet other embodiments, a patient with hemophilia A with or without inhibitors is treated with a subcutaneous dose of fitusiran at about 80 mg about every month (or about every four weeks). In yet other embodiments, a patient with hemophilia A with or without inhibitors is treated with a subcutaneous dose of fitusiran at about 20 mg about every two months (or about every eight weeks). In yet other embodiments, a patient with hemophilia A with or without inhibitors is treated with a subcutaneous dose of fitusiran at about 20 mg about every month (or about every four weeks). In yet other embodiments, a patient with hemophilia A with or without inhibitors is treated with a subcutaneous dose of fitusiran at about 10 mg about every month (or about every four weeks).

### III. Emicizumab Prophylaxis

In the present methods, the patients have been on emicizumab prophylaxis. Emicizumab is administered via subcutaneous injection about once weekly, about every other week, or about once monthly with a general dose regimen that is weight-based. The present treatment methods are for use in a patient who has been on prophylactic treatment with emicizumab. In some embodiments, the prophylactic treatment with emicizumab comprises administration of about 1.5 mg/kg to about 6 mg/kg emicizumab about once a week (about QW), about every two weeks (about Q2W), or about monthly (about QM) or about every four weeks (about Q4W). In some embodiments, the prophylactic treatment with emicizumab comprises administration of about 1.5 mg/kg QW, about 3 mg/kg Q2W, or about 6 mg/kg Q4W.

In some embodiments, the patient has been on prophylactic treatment with emicizumab at a dose of about 1.5 mg/kg emicizumab about once weekly. In some embodiments, the patient has been on prophylactic treatment with emicizumab at a dose of about 1.5 mg/kg about Q2W. In some embodiments, the patient has been on prophylactic treatment with emicizumab at a dose of about 1.5 mg/kg about once a month or about every four weeks.

In some embodiments, the patient has been on prophylactic treatment with emicizumab at a dose of about 3 mg/kg about once weekly. In some embodiments, the patient has been on prophylactic treatment with emicizumab at a dose of about 3 mg/kg about Q2W. In some embodiments, the patient has been on prophylactic treatment with emicizumab at a dose of about 3 mg/kg about once a month or about every four weeks.

In some embodiments, the patient has been on prophylactic treatment with emicizumab at a dose of about 6 mg/kg about once weekly. In some embodiments, the patient has been on prophylactic treatment with emicizumab at a dose of about 6 mg/kg about Q2W. In some embodiments, the patient has been on prophylactic treatment with emicizumab at a dose of about 6 mg/kg about once a month or about every four weeks.

In some embodiments, the patient has been on prophylactic treatment with emicizumab at a dose of about 3 mg/kg about once weekly for the first four weeks, followed by a dose of about 1.5 mg/kg about once weekly. In some embodiments, the prophylactic emicizumab treatment in the patient is terminated about one to about three months, or about four weeks to about twelve weeks, prior to the first dose of fitusiran.

In some embodiments, the prophylactic emicizumab treatment in the patient is terminated about two months or about eight weeks prior to the first dose of fitusiran; in further embodiments, this prior prophylactic emicizumab treatment is 1.5 mg/kg of emicizumab Q1W, 3 mg/kg of emicizumab Q1W, or 3 mg/kg of emicizumab Q2W.

In some embodiments, the prophylactic emicizumab treatment in the patient is terminated about two and a half months or about ten weeks prior to the first dose of fitusiran; in further embodiments, this prior prophylactic emicizumab treatment is 6 mg/kg of emicizumab Q1M.

In some embodiments, the prophylactic emicizumab treatment in the patient is terminated about six months or about six and a half months prior to any escalation in the dose of fitusiran.

### IV. Bleeding Episode Management Recommendations

The pre-fitusiran treatment period begins from the time that the final dose of emicizumab is administered. Participants will switch from emicizumab to clotting factor VIII/rFVIIa prophylaxis or on-demand therapy for the pre-fitusiran treatment period. CFC/rFVIIa prophylaxis concludes about one week, or about seven days, after the start of fitusiran prophylaxis.

During the pre-fitusiran treatment period, hemophilia A patients who are transitioning from emicizumab may receive bleed management therapy with FVIII concentrates (CFCs) or recombinant factor VIIa (rFVIIa).

During the fitusiran treatment period, the factor or BPA dose necessary to safely and effectively treat breakthrough bleeding episodes in patients receiving fitusiran will be lower than standardly prescribed. Patients may continue with their standard factor or BPA regimens for the first days following initiation of fitusiran dosing, with institution of the protocol-specific bleed management guidelines with reduced factor or BPA at Day 8 and beyond (Table 2).

TABLE 2
Bleed Management Guidelines by Product

| | | | Recombinant | |
|---|---|---|---|---|
| | Factor VIII | aPCC¹ | Factor VIIa | |
| Recommended | 10 IU/kg | 30 U/kg | ≤45 | μg/kg |
| Single dose | | | | |
| Single dose | 20 IU/kg | 50 U/kg | 45 | μg/kg |
| should not exceed | | | | |

| | | | |
|---|---|---|---|
| Repeat Dosing | Should not | Should not | Should not |
| | repeat in less | repeat in less | repeat in less |
| | than 24 hours | than 24 hours | than two hours |

¹To be used only once emicizumab levels are no longer detectable.
Abbreviations: aPCC: activated prothrombin complex concentrate; AT: antithrombin; rFVIIa: activated recombinant FVII.

More specifically, during days 1 to 7 of fitusiran treatment period, patients may continue with their standard factor or BPA regimens. After day 7 of the fitusiran treatment period, patients do not use factor, BPA, or other hemostatic agents as prophylaxis for bleeding episode prevention. At day 8 and beyond, when a patient experiences a symptom that may be consistent with bleeding episodes, the following steps are followed:
A single dose is administered according to the guidelines in Table 2.
The patient is instructed to re-evaluate symptoms in 24 hours for bleeds treated with FVIII, FIX or aPCC and in 2-3 hours for bleeds treated with rFVIIa (administration of FIX Extended half-life is not be more frequent than every 5-7 days).
Doses are not administered at less than 24-hour intervals (except IFVIIa as indicated in Table 2).
Doses do not exceed the protocol maximum recommended dose indicated in Table 2.
Antifibrinolytics are not used in combination with factor or BPA.

The bleed management guidelines in Table 2 may also be used for perioperative prophylaxis (for, e.g., perioperative bleed management of minor or major surgery). Minor surgery is defined as any invasive operative procedure in which only skin, mucous membranes, or superficial connective tissue is manipulated and does not meet the criteria for major surgery (e.g., dental extraction of <3 non-molar teeth). Major surgery is defined as any invasive operative procedure that requires any of the following:
Opening into a major body cavity (e.g., abdomen, thorax, skull),
Operation on a joint,
Removal of an organ,
Dental extraction of any molar teeth or ≥3 non-molar teeth or any tooth implantation,
Operative alteration of normal anatomy, or
Crossing of a mesenchymal barrier (e.g., pleura, peritoneum, dura).
   In participants undergoing surgery, perioperative prophylaxis may be carried out according to Table 2 and as described above.

### V. Patient Populations

The present treatments may be used to prophylactically treat patients with hemophilia A with or without inhibitors who were previously treated prophylactically with emicizumab. In some embodiments, the patient is a male. In some embodiments, the patient (e.g., a male) is an adolescent or an adult who is 12 years of age or older. In some embodiments, the patient is an adult (e.g., a male) aged ≥18 years with severe hemophilia A. A diagnosis of severe hemophilia A is based on factor VIII level of <1%. In some embodiments, the patient has had one or more (e.g., two or more, four or more, or six or more) bleeding episodes requiring factor or BPA treatment within the last six months prior to treatment.

In some embodiments, the patient has inhibitory antibodies to factor VIII (i.e., a patient with inhibitors). A patient is defined as a patient with inhibitors if they meet at least one of the following Nijmegen-modified Bethesda assay results criteria:
a. inhibitor tier of ≥0.6 BU/mL prior to treatment, or
b. inhibitor titer of <0.6 BU/mL prior to treatment with medical record evidence of two consecutive titers ≥0.6 BU/mL, or
c. inhibitor titer of <0.6 BU/mL prior to treatment with medical record evidence of anamnestic response.

In some embodiments, the patient does not have inhibitory antibodies to factor VIII (i.e., a patient without inhibitors). A patient is defined as a patient without inhibitors if they meet at least one of the following Nijmegen-modified Bethesda assay results criteria:
a. Nijmegen-modified Bethesda assay inhibitor titer of <0.6 BU/mL prior to treatment,
b. no use of bypassing agents to treat bleeding episodes for at least the last six months prior to treatment, and
c. no history of immune tolerance induction therapy within the past three years prior to treatment.

The patient has been on prophylactic treatment with emicizumab to manage bleeding episodes prior to the start of fitusiran treatment. In some embodiments, the patient has been on prophylactic treatment with emicizumab at a dose of about 6 mg/kg about once a week, about once every other week, or about once a month (or about once every four weeks). In some embodiments, the patient has been on prophylactic treatment with emicizumab at a dose of about 3 mg/kg about once a week, about once every other week, or about once a month (or about once every four weeks). In some embodiments, the patient has been on prophylactic treatment with emicizumab at a dose of about 1.5 mg/kg about once a week, about once every other week, or about once a month (or about once every four weeks).

In some embodiments, the patient does not have a known co-existing bleeding disorder other than hemophilia A, e.g., hemophilia B, von Willebrand's disease, additional factor deficiencies, acquired hemophilia, or platelet disorders.

In some embodiments, the patient does not have a history of antiphospholipid antibody syndrome or arterial or venous thromboembolism, atrial fibrillation, significant valvular disease, myocardial infarction, angina, transient ischemic attack, or stroke. Patients who have experienced thrombosis associated with indwelling venous access may be treated with the present methods.

In some embodiments, the patient has not had a malignancy within two years, except for basal or squamous cell carcinoma of the skin that has been successfully treated.

In some embodiments, the patient does not have a clinically significant liver disease, as indicated by (i) history of liver cirrhosis, portal hypertension, esophageal varices, liver fibrosis, or hepatic encephalopathy; or (ii) presence of ascites by physical exam.

In some embodiments, the patient does not have an AT activity <60% prior to treatment.

In some embodiments, the patient does not have a co-existing thrombophilic disorder, as determined by presence of any of the below:
a. FV Leiden mutation (homozygous or heterozygous),
b. protein S deficiency,
c. protein C deficiency, or
d. prothrombin mutation (G20210A; homozygous and heterozygous).

In some embodiments, the patient does not have ALT and/or AST >1.5×upper limit of normal reference range (ULN).

In some embodiments, the patient does not have total bilirubin >1.5×ULN (>2.0×ULN in patients with Gilbert's Syndrome).

In some embodiments, the patient is not Hepatitis C virus antibody positive. A patient who is Hepatitis C virus antibody positive may be treated with the present methods if they have:
a. completed curative treatment at least twelve weeks prior to enrollment and attained sustained virologic response as documented by a negative HCV RNA prior to treatment, or they have spontaneously cleared infection as documented by negative HCV RNA prior to treatment, and
b. no evidence of cirrhosis according to FibroScan <12.5 kPa (where available), or FibroTest score <0.75 and APRI <2 (if FibroScan unavailable).

In some embodiments, the patient does not have acute hepatitis, i.e., hepatitis A or hepatitis E.

In some embodiments, the patient does not have acute or chronic hepatitis B infection (IgM anti-HBc antibody positive or Hepatitis B serum antigen positive).

In some embodiments, the patient does not have a platelet count≤100,000/μL.

In some embodiments, the patient is not HIV positive with CD4 count <200 cells/μL.

In some embodiments, the patient does not have an estimated glomerular filtration rate <45 mL/min/1.73 m² (according the Modification of Diet in Renal Disease [MDRD] formula).

In some embodiments, the patient meets one or more of the inclusion criteria and one or more of the exclusion criteria detailed in Example 1 below.

### VI. Treatment Outcomes and Evaluation

In some embodiments, the present treatment methods reduce the frequency of bleeding episodes in a patient. A bleeding episode is defined as any occurrence of hemorrhage that requires replacement factor or BPA infusion, e.g., hemarthrosis, muscle, or mucosal bleeding. The definition of bleeding episode types described below is based on consensus opinion of International Society on Thrombosis and Haemostasis (ISTH) (Blanchette et al., J Thromb Haemost. (2014) 12 (11): 1935-9).

The start time of a bleeding episode is defined as the time at which symptoms of a bleeding episode first develop. Bleeding or any symptoms of bleeding at the same location that occurs within 72 hours of the last injection used to treat a bleeding episode at that location is considered a part of the original bleeding event, and will count as one bleeding episode towards the ABR. Any bleeding symptoms that begin more than 72 hours from the last injection used to treat a bleeding episode at that location will constitute a new bleeding event.

A spontaneous bleeding episode refers to a bleeding event that occurs for no apparent or known reason, particularly into the joints, muscles, and soft tissues. A joint bleeding episode is characterized by an unusual sensation in the joint (“aura”) in combination with 1) increasing swelling or warmth over the skin over the joint, 2) increasing pain, or 3) progressive loss of range of motion or difficulty in using the limb as compared with baseline.

A muscle bleed may be characterized by pain, swelling and loss of movement over the affected muscle group. A target joint is defined as a joint where three or more spontaneous bleeding episodes in a single joint within a consecutive six-month period has occurred; where there have been less than or equal to two bleeding episodes in the joint within a consecutive 12-month period the joint is no longer considered a target joint. A traumatic bleeding episode is one that is caused by a known injury or trauma. Bleeding episodes sustained during sports and recreation is counted as traumatic bleeding episodes.

In some embodiments, the present treatment methods result in reduction in the annualized bleed rate (ABR), and/or the annualized spontaneous bleeding rate (AsBR) in the treated population compared to a population treated prophylactically with compared to another prophylaxis treatment (e.g., replacement factor, BPA, or emicizumab).

In some embodiments, the present treatment methods result in improved patient-reported outcomes (PROs) or an improvement in quality of life (QoL) as further described below. Hemophilia, through its associated symptoms, functional limitations and treatment burden, directly impacts the health-related quality of life (HRQOL) of patients. Improving HRQOL is a critical aspect of hemophilia disease management. The present methods improve HRQOL of patients as determined by well-designed, detailed questionnaires.

In some embodiments, HRQOL is measured by the Patient-Reported Outcomes Measurement Information System v2.0 Pain Intensity 3a (PROMIS v2.0 Pain Intensity 3a). The PROMIS v2.0 Pain Intensity 3a is a system of reliable and precise measures of adolescent and adult (age at least twelve years) participant-reported health status (Cella et al., Med Care. (2007) 45 (5 Suppl 1): S3-S11). The PROMIS v2.0 Pain Intensity 3a is a psychometrically and clinically tested instrument assessing how much a person hurts. The instrument consists of three items measuring the worst, average, and current intensity of pain with a recall period of 7 days. Each question has five response options ranging from one (“no pain”) to five (“very severe”). Raw scores are translated into a T-score (an item-level calibration adjustment). A higher PROMIS T-score represents more of the concept being measured (Pain Intensity). The questionnaire may be taken following the final administration of emicizumab and after treatment with one or more (e.g., two or more, three or more, four or more, five or more, or six or more) doses of fitusiran (e.g., administered subcutaneously at 50 mg once every four weeks or once a month). For example, the questionnaire may be taken at about month 6, 12, or 18 after commencement of fitusiran treatment. In some embodiments, the present treatment methods decrease the PROMIS T-score compared to another prophylaxis treatment.

In some embodiments, the improvement in PRO or in QoL is measured using the Treatment Satisfaction Questionnaire for Medication-9 Items (TSQM-9). The TSQM-9 assesses participants' satisfaction with treatment. The TSQM-9 is a psychometrically validated tool that provides a general measure of participants' satisfaction with medication (Atkinson et al., Health Qual Life Outcomes. (2004) 2:12). The questionnaire consists of nine items directed to the overall patient satisfaction or dissatisfaction with the medication administered in the clinical trial, as measured by the patient evaluation of the effectiveness, side effects, and convenience of the medication over the preceding two to three weeks. Each question has seven response options ranging from one (“extremely dissatisfied”) to seven (“extremely satisfied”). Higher scores indicate better health status. The questionnaire may be taken following the final administration of emicizumab and after treatment with one or more (e.g., two or more, three or more, four or more, five or more, or six or more) doses of fitusiran (e.g., administered subcutaneously at about 50 mg once about every four weeks or about once a month). For example, the questionnaire may be taken at about month 6, 12, or 18 after commencement of fitusiran treatment. In some embodiments, the present treatment methods increase the TSQM-9 score compared to another prophylaxis treatment.

In some embodiments, the improvement in PRO or in QoL is measured using the Preference Questionnaire. The Preference Questionnaire is a three-item questionnaire measuring participants' preferences for fitusiran compared to standard of care. The Preference Questionnaire evaluates the respondent's preferred treatment, reasons for preference, and willingness to continue treatment. The questionnaire consists of three items directed to the patient preference for fitusiran versus emicizumab, the main reasons for the indicated preference, and the respondent's willingness to continue their preferred treatment method. Respondents indicate preference for either emicizumab (prior to fitusiran treatment), the fitusiran treatment administered during the clinical trial, or no preference for either treatment. Respondents rank the three main reasons for their preferred treatment selected from the following: less time required to receive the treatment, less frequent administration, easier to use, more flexibility in treatment administration, better quality of life, or less worry about having a bleed. The respondent's willingness to continue their preferred treatment is indicated by a range of five response options from “extremely unwilling” to “extremely willing.” The questionnaire may be taken twelve months after commencement of fitusiran treatment. In some embodiments, the present treatment methods increase the reported preference for fitusiran prophylaxis compared to another prophylaxis treatment.

In some embodiments, the improvement in PRO or in QoL is measured using the International Physical Activity Questionnaire (IPAQ). The IPAQ measures levels of physical activity in adult participants (≥18 years) by collecting information on household and yard work activities, occupational activity, self-powered transport, and leisure time physical activity as well as sedentary activity (Craig et al., Med Sci Sports Exerc. (2003) 35 (8): 1381-95). The questionnaire consists of 27 items directed to the number of days and amount of time spent sitting, walking, and engaging in moderate and vigorous physical activities in the last seven days or the last usual week. Global and domain scores can be calculated with higher scores denoting higher physical activity. The questionnaire may be taken following the final administration of emicizumab and after treatment with one or more (e.g., two or more, three or more, four or more, five or more, or six or more) doses of fitusiran (e.g., administered subcutaneously at 50 mg about once every four weeks or about once a month). For example, the questionnaire may be taken at about month 6, 12, or 18 after commencement of fitusiran treatment. In some embodiments, the present treatment methods increase the IPAQ score compared to another prophylaxis treatment.

In some embodiments, the improvement in PRO or in QoL is measured using participant qualitative interviews. Patient qualitative interviews document the experience of treatment and trial participation from the patient perspective. Specifically, the interviews capture 1) experience of living with hemophilia, 2) experience with treatments prior to the trial participation, and 3) expectations for the trial and experiences during the trial. The interview may be conducted within two weeks after participant screening and within two weeks of the end of the study. Dominant trends can be identified in each interview and compared across the results of the other interviews to generate themes or patterns in the way participants describe their disease, treatment, and clinical trial experiences. In some embodiments, the present treatment methods produce a favorable experience with fitusiran prophylaxis compared to another prophylaxis treatment

In some embodiments, the present treatment methods improve the joint health of patients with hemophilia. The efficacy of fitusiran in improving joint health is measured using the Hemophilia Joint Health Score (HJHS). The HJHS is a clinician-reported outcome tool for the evaluation of joint health in participants with hemophilia. The HJHS assesses impairment in six joints (right and left elbows, knees, and ankles) and includes a global gait score assessed by a trained physician or trained healthcare professional using an 11-item score (swelling, duration of swelling, axial alignment, muscle atrophy, crepitus, flexion loss, extension loss, joint pain, instability, gait, and strength). Total scores range from 0 to 148, with lower scores denoting less functional limitations (reduced impairment). The HJHS assessment may be administered following the final administration of emicizumab, at the time of the first dose of fitusiran prophylaxis, and after treatment with one or more (e.g., two or more, three or more, four or more, five or more, or six or more) doses of fitusiran (e.g., administered subcutaneously at 50 mg administered about once every four weeks or about once a month). In some embodiments, the present treatment methods reduce the HJHS score of patients treated with fitusiran prophylaxis compared to another prophylaxis treatment.

According to the International Conference on Harmonisation (ICH) E2A guideline Definitions and Standards for Expedited Reporting, and 21 Code of Federal Regulations (CFR) 312.32, IND Safety Reporting, an adverse event (AE) is any untoward medical occurrence in a patient or clinical investigational patient administered a medicinal product and which does not necessarily have a causal relationship with this treatment. Since bleeding episodes are recorded as an efficacy assessment of fitusiran, these will not be treated as AEs unless they meet any of the severe adverse event (SAE) criteria listed below.

An SAE is any untoward medical occurrence that at any dose:
a. results in death,
b. is life-threatening (an event which places the patient at immediate risk of death from the event as it occurred. It does not include an event that had it occurred in a more severe form might have caused death),
c. requires in-patient hospitalization or prolongation of existing hospitalization,
d. results in persistent or significant disability or incapacity,
e. is a congenital anomaly or birth defect, or
f. is an important medical event that may not be immediately life-threatening or result in death or hospitalization but may jeopardize the patient and may require intervention to prevent one of the other outcomes listed in the definition above (e.g., events include allergic bronchospasm requiring intensive treatment in an emergency room or at home, blood dyscrasias, convulsions, or the development of drug dependency or abuse).

### VII. Articles of Manufacture and Kits

The present invention also provides kits comprising a pharmaceutical composition for use in the present treatment methods. Such kits include one or more vials or one or more pre-filled syringes or injectors (e.g., pens) comprising a pharmaceutical composition of the invention and instructions for use, e.g., instructions for administering a therapeutically effective amount of fitusiran.

In some embodiments, fitusiran is packaged in a drug delivery device. In some embodiments, a drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. As also described in ISO 11608-1:2014(E), a single-dose container system may involve a needle-based injection device with an integrated non-replaceable container. In some embodiments, each container holds a single dose, whereby the entire deliverable volume is expelled (full evacuation). In other embodiments, each container holds a single dose, whereby a portion of the deliverable volume is expelled (partial evacuation).

An exemplary sleeve-triggered auto-injector with manual needle insertion is described in WO2015/004052. Examplery audible end-of-dose feedback mechanisms are described in WO2016/193346 and WO2016/193348. An exemplary needle-safety mechanism after using an auto-injector is described in WO2016/193352. An exemplary needle sheath remover mechanism for a syringe auto-injector is described in WO2016/193353. An exemplary support mechanism for supporting an axial position of a syringe is described in WO2016/193355.

Fitusiran may be supplied in a single-use vial containing 20 mg fitusiran (equivalent to 21 mg fitusiran sodium) in 0.2 mL of solution at a concentration of 100 mg/mL. Fitusiran may also be supplied in a pre-filled pen containing 50 mg of fitusiran (equivalent to 53 mg fitusiran sodium) in 0.5 mL solution at a concentration of 100 mg/mL.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of hematology, medicine, medicinal and pharmaceutical chemistry, and cell biology described herein are those well-known and commonly used in the art. Further, unless otherwise required by context, singular terms shall include pluralities and plural terms shall include the singular. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain aspects, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Clinical Trial Protocol for an Open-Label, Single Arm Treatment Study to Investigate the Safety and Tolerability of Switching from Emicizumab to Fitusiran Prophylaxis in Male Participants Aged ≥18 Years of Age with Severe Hemophilia A, with or without Inhibitors

The Example describes an open-label, single-arm treatment Phase 1 study to investigate the safety and tolerability of fitusiran prophylaxis in adult male patients with severe hemophilia A with or without inhibitory antibodies to Factor VIII (FVIII) who have previously received emicizumab prophylaxis. The primary objective of the study is to characterize the safety of starting fitusiran approximately two months or two and a half months after ceasing emicizumab treatment.

Approximately two months after terminating emicizumab treatment, fitusiran prophylactic treatment will be initiated at a regimen of 20 mg Q2M with the aim for gradual increase in thrombin generation (TG) potential while emicizumab residual levels continue to decrease. This treatment regimen is chosen to ensure a sustained TG potential before the next fitusiran dosing and to consolidate protection against bleeding after fitusiran introduction. A dose of 20 mg fitusiran Q2M is the lowest dose investigated in adults that has the potential to reach AT activity level in the desired range of 15% to 35%.

### Justification for Dose

A population pharmacokinetic/pharmacodynamic (pop PK/PD) model that describes the dynamics of AT activity level for participants treated with fitusiran was developed to find dosing regimens that could maintain AT activity levels between 15% and 35% as a risk mitigation strategy for vascular thrombotic events. The model was characterized on AT activity data from Phase 1, Phase 2, and Phase 3 studies. The model with the estimated parameters was used to simulate AT activity level at steady state for different dosing scenarios in a virtual population of 1000 participants.

The starting dose for all participants in this study will be 20 mg Q2M. Four months after the first dose of fitusiran, participants who have either AT levels greater than 35% or who require escalation for clinical reasons will be eligible to escalate to 50 mg Q2M. Based on the simulation results, at the 50 mg Q2M regimen, approximately 45.7% of the virtual participants are expected to have AT activity <15% and therefore may require a de-escalation in dose. Per protocol, these participants will need to de-escalate from 50 mg Q2M to 20 mg Q2M.

The simulations for lower dosing regimens in 1000 de-novo virtual participants, namely 20 mg Q2M and 20 mg QM, predict that 3.8% and 23.4% of the participants, respectively, would have AT activity level <15%. Thus, based on the simulations, 20 mg Q2M was considered an appropriate de-escalation regimen for the 45.7% participants who may have AT activity level <15% at 50 mg Q2M. On de-escalation to 20 mg Q2M, approximately 8% of the de-escalated participants may have AT activity level <15% and approximately 7% of the de-escalated participants may have AT activity level >35%, while the rest, 84% to 85%, of de-escalated participants are predicted to have AT activity level within the target AT window of 15% to 35%.

### Duration of Treatment

The duration of treatment with fitusiran is 18 months. The estimated total time of the study is up to 28 months. The estimated total time includes a two-month screening period, a two-month washout period following the final dose of emicizumab, an 18-month treatment period with fitusiran, and a six-month follow-up period after the final dose of fitusiran to monitor AT activity levels. For participants who receive emicizumab on a QW or Q2W schedule, the washout period will be two months. For participants who receive emicizumab on a monthly schedule, the washout period will be 2.5 months.

### Study Objectives and Endpoints

The primary objective of this study is to describe the safety and tolerability of switching from emicizumab prophylaxis to fitusiran prophylaxis after a transition period.

The secondary objectives of this study are:
to evaluate the peak thrombin generation (TG) profile and antithrombin (AT) levels during fitusiran prophylaxis throughout the six-months after the final dosed of emicizumab;
to evaluate the emicizumab levels for up to six months following the final dose of emicizumab;
to describe the safety and tolerability of switching from emicizumab prophylaxis to fitusiran prophylaxis after transition period;
to characterize the change from baseline in the following outcomes related to participants' Health-Related Quality of Life (HRQOL) after switching to fitusiran:
   participant overall satisfaction with fitusiran as well as assessment of pain of injections with both therapies to measure treatment and injection burden,
   participant treatment preference,
   participant pain intensity, and
   participant physical functioning and physical activity;
to characterize the participants' joint health after switching to fitusiran as assessed by the physician; and
to characterize the frequency of treated bleeding episodes while receiving fitusiran prophylaxis.

The exploratory objective is to characterize the participants' overall experiences. The primary endpoint of this study is to evaluate the incidence, severity, and

seriousness of adverse events (AEs) from D1 to M4 of fitusiran treatment.

The secondary endpoints are to evaluate:
peak TG and AT levels from the final dose of emicizumab to the first day of the pre-fitusiran washout period, and from D1 to M4 of the fitusiran treatment period;
emicizumab concentrations in plasma up to complete washout (approximately M4) of fitusiran treatment;
incidence, severity, and seriousness of AEs from DI to M18 of fitusiran treatment through:
   change in participants' overall satisfaction during the fitusiran treatment period assessed via the Treatment Satisfaction Questionnaire for Medication (TSQM-9) domain over time from baseline until EOS,
   participants' treatment preferences (via the Preference Questionnaire),
   change in participants' pain intensity during the fitusiran treatment period (via PROMIS Pain Intensity 3a questionnaire) over time, from baseline until EOS, and
   change in participants' physical functioning and physical activity during the fitusiran treatment period (via the IPAQ) over time, from baseline until EOS;
change in participants' joint health (via the Hemophilia Joint Health Score [HJHS]) during the fitusiran treatment period over time, from baseline until EOS; and
Annualized Bleeding Rate (ABR) while receiving fitusiran prophylaxis during the 14-month extension period.

The exploratory endpoint is characterization of the participants' overall experience via participant exit interviews.

### Study Design

The study evaluates male patients, aged ≥18 years, with severe hemophilia A with or without inhibitory antibodies to FVIII who have switched from prior standard of care with emicizumab prophylaxis.

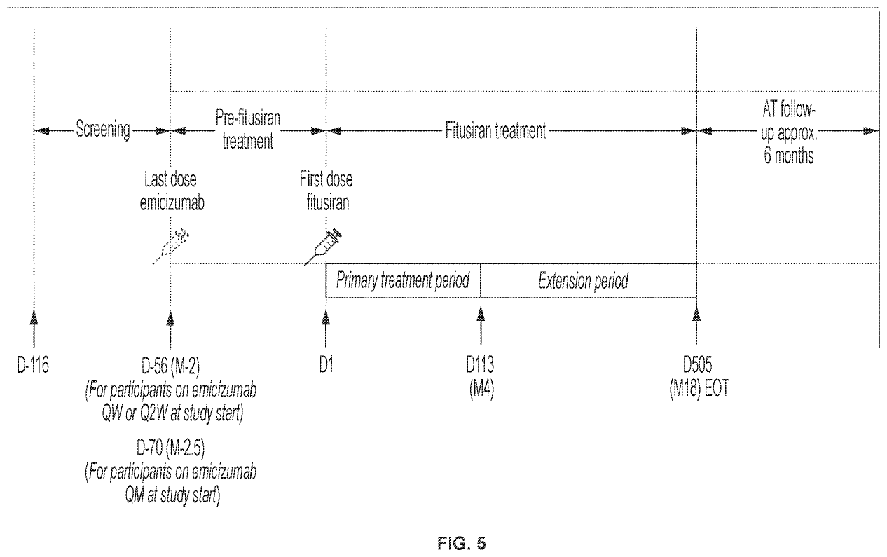

Participants complete screening during an up to two-month screening period. Thereafter, the study has three study periods: an approximately two-month pre-fitusiran treatment period, an 18-month fitusiran treatment period, and an antithrombin follow-up (AT FU) period (FIG. 5).

During the approximately two-month pre-fitusiran treatment period from the final dose of emicizumab, participants will continue their prior regiment with CFCs or BPAs. For study participants who were on emicizumab QM at study start, the pre-fitusiran treatment period starts with their last dose of emicizumab on Day −70 (month −2.5) and ends on Day −1. For study participants who were on emicizumab Q2W or QW at study start, the pre-fitusiran treatment period starts with their last dose of emicizumab on Day −56 (Month −2) and ends on Day −1. Participants receiving emicizumab (Hemlibra) at screening will switch to CFC/BPA prophylaxis or on-demand therapy for the pre-fitusiran treatment period as they cannot remain on emicizumab on and after Day −70 or −56 (depending on emicizumab administration schedule). For participants who received emicizumab, bleed- and dosing-related data will be collected from medical records from the six-month period prior to screening.

During the 18-month fitusiran treatment period, participants will receive fitusiran prophylaxis (Day 1 to Day 505). Study participants may need to adjust their fitusiran regimen (dose/frequency) based on individual participant response. Dose adjustments may occur as summarized in FIG. 3. The primary treatment period will be from Day 1 to Day 113. The efficacy extension period will occur between Day 114 to Day 533 or 561 (depending on the fitusiran dosing frequency of QM or Q2M).

After completion of the treatment period or premature permanent discontinuation of fitusiran prophylaxis, participants will enter an antithrombin follow-up (AT FU) period, during which the AT activity level will be monitored (at approximately monthly intervals following the final fitusiran dose and/or the ET visit) until AT activity levels return to at least 60% as per the central laboratory. If applicable, for participants continuing fitusiran prophylaxis directly after the trial, the AT follow-up visits are not required.

Breakthrough bleeding events and doses or CFCs and BPAs administered after enrollment will be recorded. Safety, QoL, and PD data will also be collected.

During the pre-fitusiran treatment period, participants previously receiving emicizumab (Hemlibra) will switch to CFC/BPA prophylaxis or on-demand therapy. Throughout the study, all participants may receive episodic/on-demand treatment for breakthrough bleeding episodes with CFC or BPAs, as appropriate. A summary of the study periods is presented in Table 3.

TABLE 3
Study Periods

| | | | Associated |
|---|---|---|---|
| Period | | | Intervention |
| Title | Period Type | Period Description | Labels |
| Pre- | Other | The pre-fitusiran treatment period is | Emicizumab |
| fitusiran | | defined as the period after the last dose | CFC/BPA |
| treatment | | of emicizumab up to the first fitusiran | (rFVIIa) |
| period | | administration. Participants will receive | |
| | | on demand or prophylactic treatment with | |
| | | IV CFCs or BPAs from M −2 until D 1. | |
| | | In case of bleeding events participants | |
| | | will receive IV CFCs or BPAs. | |
| Fitusiran | Experimental | Participants will receive SC fitusiran | Fitusiran |
| treatment | | prophylaxis Q2M or QM from D 1 until | CFC/BPA |
| period | | M 18. | (rFVIIa) |
| | | In case of bleeding events participants | ATIIIC |
| | | will receive IV CFCs or BPAs. | |
| | | Participants may receive IV ATIIIC at | |
| | | Investigator discretion. | |
| AT | Other | Participants will be followed up until AT | CFC/BPA |
| Follow-Up | | activity levels recover to at least 60% | (rFVIIa) |
| period | | (per central laboratory). | ATIIIC |
| | | In case of bleeding events participants | |
| | | will receive IV CFCs or BPAs. | |
| | | Participants may receive IV ATIIIC as | |
| | | rescue medicine. | |

Abbreviations: AT: antithrombin; ATIIIC: antithrombin concentrate; BPA: bypass agent; CFC: clotting factor concentrate; QM: every month; Q2M: every two months; rFVIIa: activated recombinant factor VII; SC: subcutaneous.

### Study Population

Participants are eligible to be included in the study if all of the following criteria apply:

I 01. Male participants must be ≥18 years of age inclusive, at the time of signing the informed consent.

I 02. Diagnosis of severe congenital hemophilia A (FVIII <1%) as evidenced by a central laboratory measurement at Screening or documented medical record evidence. Inhibitor titer of ≥0.6 BU/mL at Screening, or Inhibitor titer of <0.6 BU/mL at Screening with medical record evidence of two consecutive titers ≥0.6 BU/mL, or Inhibitor titer of <0.6 BU/mL at Screening with medical record evidence of anamnestic response.

I 03. Participants who are currently on the full labeled dose (6 mg/kg) of emicizumab prophylaxis, irrespective of inhibitor/non-inhibitor status.

I 04. Participants using rFVIIa or willing to switch to rFVIIa as primary BPA for the treatment of breakthrough bleeds from Day-56 to M4 after the start of fitusiran treatment.

I 05. Male.

Participants are to be excluded from the study if any of the following criteria apply:

### Medical Conditions

E 01. Known coexisting bleeding disorders other than congenital hemophilia A, i.e., hemophilia B, von Willebrand disease, additional factor deficiencies, acquired hemophilia, or platelet disorders.

E 02. History of antiphospholipid antibody syndrome.

E 03. History of arterial or venous thromboembolism, atrial fibrillation, significant valvular disease, myocardial infarction, angina, transient ischemic attack, or stroke. Participants who have experienced thrombosis associated with indwelling venous access may be enrolled.

E 04. History of malignancy within two years prior to Screening, except for basal or squamous cell carcinoma of the skin that has been successfully treated.

E 05. Completion of a surgical procedure within 14 days prior to screening, or currently receiving additional CFC or BPA infusion for postoperative hemostasis.

E 06. At Screening, anticipated need of surgery during the study or planned surgery scheduled to occur during the study.

E 07. History of intolerance to SC injection(s).

E 08. History of alcohol abuse within the twelve months before Screening. Alcohol abuse in this study is defined as regular weekly intake of more than 14 units (1 unit=1 glass of wine [approximately 125 mL]=1 measure of spirits (approximately 1 fluid ounce)=½ pint of beer [approximately 284 mL]).

E 09. Presence of clinically significant liver disease or any of the following conditions:
history of liver cirrhosis, portal hypertension, esophageal varices, liver fibrosis, or hepatic encephalopathy or
presence of ascites by physical examination.

### Prior/Concomitant Therapy

E 11. Good control of bleedings and good drug-tolerance (no side-effects) under emicizumab.

E 12. Current participation in immune tolerance induction therapy.

E 13. Prior gene therapy.

### Prior/Concurrent Clinical Study Experience

E 14. Current or prior participation in a fitusiran trial.

E 15. Current or prior participation in a gene therapy trial.

E 16. Received an investigational drug or device within 30 days prior to the Screening Visit or within five half-lives of the investigational drug (or devices containing investigational medicinal product [IMP]) prior to the Screening Visit, whichever is longer.

### Diagnostic Assessments

E 17. AT activity <60% at Screening, as determined by central laboratory measurement.

E 18. Coexisting thrombophilic disorder, as determined by presence of any of the following as identified at central laboratory (or via historical results, where available):
Factor V Leiden mutation (homozygous or heterozygous).
Protein S deficiency.
Protein C deficiency.
Prothrombin mutation (G20210A; homozygous or heterozygous).

E 19. ALT and/or AST >1.5×upper limit of normal (ULN) reference range.

E 20. Total bilirubin >1.5×ULN (>2.0×ULN in participants with Gilbert's Syndrome).

E 21. Hepatitis C virus (HCV) antibody positive, except participants with a history of HCV infection who meet both conditions below:
Completed curative treatment at least twelve weeks prior to enrollment and attained sustained virologic response as documented by a negative HCV ribonucleic Acid (RNA) at screening, or they have spontaneously cleared infection as documented by negative HCV RNA at Screening.
No evidence of cirrhosis according to one of the following assessments:
   FibroScan <12.5 kPa (where available), or,
   FibroTest score <0.75 and AST-to-platelet ratio index (APRI)<2 (if FibroScan unavailable).

E 22. Presence of acute hepatitis, i.e., hepatitis A, hepatitis E.

E 23. Presence of acute or chronic hepatitis B infection (IgM anti-HBc antibody positive or Hepatitis B serum antigen (HBs Ag) positive).

E 24. Platelet count ≤100,000/μL.

E 25. Presence of acute severe or serious infection at Screening.

E 26. Known to be Human Immunodeficiency Virus (HIV) positive with CD4 count <200 cells/μL.

E 27. Estimated glomerular filtration rate ≤45 mL/min/1.73 m² (using the Modification of Diet in Renal Disease formula).

### Study Interventions

Study interventions are all pre-specified, investigational, and non-investigational medicinal products, medical devices, and other interventions (e.g., surgical and behavioral) intended to be administered to the study participants during the study conduct. Study participants will receive their last dose of emicizumab on D-56 and will start fitusiran prophylaxis on D1. Study treatments and modes of administration are summarized in Table 4 below.

TABLE 4
Study Treatments

| | | Clotting factor | | |
|---|---|---|---|---|
| | | concentrate | | |
| | | (CFC) or | Antithrombin | |
| | Fitusiran | bypassing agents | concentrate | |
| | (SAR439774) | (BPA) | (ATIIIC) | Emicizumab |
| Intervention | Participants will | Participants will | Participants may | Participants will be |
| Description | be administered | receive IV CFCs | receive IV | administered their |
| | fitusiran as an | or FFVIIa | ATIIIC at | last dose of |
| | SC injection QM | during the pre- | Investigator | emicizumab |
| | or Q2M. | fitusiran treatment | discretion. | approximately |
| | | period or in | Antithrombin III | two months before |
| | | case of bleeding | (ATC | the first dose |
| | | events throughout | code: B01AB02) | of fitusiran. |
| | | the study. | ATIIIC treatment | Emicizumab |
| | | The following | should follow | (ATC code: B02BX06) |
| | | treatments should | local labeled | Emicizumab is a |
| | | be used: | product | prohibited |
| | | Coagulation FVIII | recommendations | medication during |
| | | Coagulation | for the | the study |
| | | factor VIIa | prevention of | after Day −70 (for |
| | | FVIII inhibitor | perioperative | participants |
| | | bypassing activity | thrombosis in | on QM dosing at |
| | | CFC/BPA doses | participants with | study start) |
| | | and frequency | AT deficiency. | or Day −56 (for |
| | | should follow the | | participants on |
| | | local prescribing | | Q2W/QW dosing at |
| | | information during | | study start) |
| | | the pre-fitusiran | | |
| | | treatment period. | | |
| Type | Drug | Biological/Vaccine | Biological/Vaccine | Biological/Vaccine |
| Dose | Solution for | Solution for | Solution for | Solution for |
| Formulation | injection | injection | injection | injection |
| Unit dose | 100 mg/mL | Depends on | Depends on | Depends on |
| strength | | pharmaceutical | pharmaceutical | pharmaceutical |
| | | presentation | presentation | presentation |
| Dosage levels | 20 mg (S2M | Depends on | Depends on | 1.5 mg/kg, 3 |
| | or QM), | pharmaceutical | pharmaceutical | mg/kg, or 6 mg/kg. |
| | 50 mg (Q2M | presentation | presentation | Dose level is |
| | or QM), | | | determined by the |
| | 80 mg (QM) | | | participant's dose |
| | | | | regimen at study |
| | | | | start |
| Route of | SC injection | IV injection | IV injection | SC injection |
| Administration | | | | |
| Use | Experimental | Other | Other | Background |
| | | | | intervention |
| IMP and AxMP | IMP | AxMP | AxMP | AxMP |
| Packaging and | Study intervention | Study intervention | Study intervention | |
| Labeling | will be provided | will be provided | will be provided | |
| | in vials (for | in vails packed in | in vials packed in | |
| | dosing of 80 mg, | a carton box. | a carton box. | |
| | 50 mg, and 20 mg) | | | |

Abbreviations: AT: antithrombin; ATIIIC: antithrombin concentrate; AxMP: auxiliary medicinal product; BPA: bypassing agent; CFC: clotting factor concentrate; IMP: investigational medicinal product; IV: intravenous(ly); FVIII: factor VIII; QM: once monthly; rFVIIa: recombinant factor VII; SC: subcutaneous(ly).

### Dosing Regimens and Formulation

Fitusiran is formulated as a 100 mg/mL sterile solution in vials for subcutaneous injection in phosphate buffered saline (PBS). Patients will receive 20 mg Q2M or QM, 50 mg Q2M or QM, or 80 mg QM for a total of 18 months.

### Dose Modification

All study participants will start fitusiran treatment at a dose of 20 mg Q2M. The first dose will be administered on D1. Participants may escalate to 50 mg Q2M starting from Month 4 and switch to monthly dosing depending on dose de-escalation or escalation criteria targeting an AT activity range of 15%-35% (as per central laboratory). Clinical criteria are also defined for dose adjustments based on Investigator's judgment on the bleed prevention achieved at each dose level.

If an AE occurs in a participant that the Investigator judges as presenting a potential risk to the participant for further dosing, fitusiran dosing may be held at the discretion of the Investigator and the Medical Monitoring Team may be contacted.

Antithrombin Criteria for Dose Adjustment: Study participants experiencing AT activity levels outside of the target range (<15% or >35% as per central laboratory) must follow the instructions outlined below.

De-escalation in Case of Antithrombin <15%: Study participants must de-escalate or discontinue fitusiran if they experience more than one AT activity level <15% (as per central laboratory) within a 12-month period at their current dose level.

At each dose level, upon the first AT activity level <15% (as per central laboratory), the participant must have another AT measurement within one week of site receipt of the results. If the result is <15% (as per central laboratory), this will be considered the second AT level <15%. Participants with one AT levels <15% must not receive fitusiran at the current regimen until the AT activity level from the second measurement is available to guide management.

Participants receiving fitusiran once every two months (Q2M):
Participants receiving fitusiran at a dose of 20 mg Q2M who experience more than one AT activity level <15% (as per central laboratory) within a 12-month period must permanently discontinue fitusiran treatment.
Participants receiving fitusiran at a dose of 50 mg Q2M who experience more than one AT activity level <15% (as per central laboratory) within a 12-month period must be de-escalated to 20 mg QM. Participants may resume dosing at the monthly visit immediately after the participant's AT activity level has recovered to ≥22% (as per central laboratory).

Participants receiving fitusiran once monthly (QM):
Participants previously escalated to a dose of 50 mg QM or 80 mg QM due to AT ≥35% who experience more than one AT activity level <15% (as per central laboratory) within a 12-month period at their current escalation dose level:
   must either permanently discontinue fitusiran treatment, or
   may be de-escalated to their prior dose level, i.e., to 50 mg Q2M (from 50 mg QM) or to 50 mg QM (from 80 mg QM). Further dose escalation attempts are not permitted for these participants. Dosing at the lower dose level can be resumed as per visit schedules Q2M or QM when the participant's AT activity level has recovered to ≥22% (as per central laboratory).
Participants receiving fitusiran at a dose of 20 mg QM who experience more than one AT activity level <15% (as per central laboratory) within a 12-month period at their current escalation dose level must permanently discontinue fitusiran treatment.
Participants previously escalated to a dose of 50 mg QM or 80 mg QM based upon clinical criteria who experience more than one AT activity level <15% (as per central laboratory) within a 12-month period at their current escalation dose level must permanently discontinue fitusiran treatment.

### Escalation in Case of AT >35%

Participants are eligible for dose escalation if:
at least four doses of fitusiran have been administered at the current dose level, and
they have experienced at least two AT activity levels >35% (as per central laboratory) starting with the AT activity measurement at their third dosing visit at the current dose level (except for starting 20 mg Q2M dose regimen during which participants can escalate after two fitusiran doses, so starting from Month 4), and fitusiran administration and AT activity assessments occurred as per schedule at the current dose level.

Eligible participants receiving fitusiran at
a dose of 20 mg Q2M are to be escalated to 50 mg Q2M.
a dose of 50 mg Q2M are to be escalated to 50 mg QM.
a dose of 50 mg QM are to be escalated to 80 mg QM.

When escalating from Q2M to QM (50 mg) dosing schedule, the last Q2M dose will also be considered as the first dose of QM dosing.

Clinical Criteria for Dose Adjustment: The Investigator may escalate the study participant to a higher dose of fitusiran, despite an AT activity level≤35%, if at the current dose level at least two doses of fitusiran have been administered, and the Investigator judges suboptimal bleeding control, defined as two or more bleeds treated with CFC or BPA within a 12-week period. For participants treated with QM dosing, bleeding episodes during the first eight weeks at the current dose level will not be considered for this judgment. For participants treated with Q2M dosing, bleeding episodes during the first twelve weeks at the current dose level will not be considered for this judgment.

Antithrombin activity levels and additional clinical data, as applicable, will be considered in the dose escalation of individual participants.

### Study Assessments

A bleeding episode is defined as any occurrence of hemorrhage that requires administration of CFCs or BPAs, e.g., hemarthrosis, muscle, or mucosal bleeding. The definition of bleeding episode types described below is based on consensus opinion of the International Society of Thrombosis and Haemostasis (ISTH) (Irio et al., Ann Intern Med (2019) 171(8): 540-6).

The start time of a bleeding episode will be considered the time at which symptoms of a bleeding episode first develop. Bleeding or any symptoms of bleeding at the same location that occur within 72 hours of the last injection used to treat a bleeding episode at that location will be considered a part of the original bleeding event, and will count as one bleeding episodes towards the ABR. Any bleeding symptoms that begin more than 72 hours from the last injection used to treat a bleeding episode at that location will constitute a new bleeding event.

A spontaneous bleeding episode is a bleeding event that occurs for no apparent or known reason, particularly into the joints, muscles, and soft tissues.

A joint bleeding episode is characterized by an unusual sensation in the joint (“aura”) in combination with 1) increasing swelling or warmth over the skin over the joint, 2) increasing pain, or 30 progressive loss of range of motion or difficulty in using the limb as compared with baseline.

A muscle bleed may be characterized by pain, swelling and loss of movement over the affected muscle group.

A target joint is defined as a joint where three or more spontaneous bleeding episodes in a single joint within a consecutive six-month period have occurred; where there have been ≤2 bleeding episodes in the joint within a consecutive 12-month period the joint is no longer considered a target joint.

A traumatic bleeding episode is one that is caused by a known injury or trauma. Bleeding episodes sustained during sports and recreation will be counted as traumatic bleeding episodes, but participants will be asked to indicate in the eDiary that the event occurred during such activities.

Patient-reported outcomes (PROs) will be utilized in this study to assess health-related quality of life (HRQOL), physical activity, and treatment satisfaction.

### Management of Bleeding Episodes During the Pre-Fitusiran Treatment Period

During the pre-fitusiran treatment period all participants should receive bleed management therapy with FVIII concentrates (CFCs) or rFVIIa as approved locally. Local prescribing information should be followed when treating participants with prior emicizumab treatment.

### Management of Breakthrough Bleeding Episodes During the Fitusiran Treatment Period

Participants on fitusiran will be provided a written breakthrough bleed management plan with appropriate dosing of CFC or IFVIIa for use during day 1 through day 7, as well as a bleed management plan with dosing for day 8 and beyond. Thereafter the breakthrough bleed management plan will be reviewed and updated at monthly visits, and new written plans provided to the participant if dosing changes.

After day 7, participants receiving fitusiran prophylaxis as per protocol should not use CFC, BPA (e.g., FVIIa), or other hemostatic agents as prophylaxis or bleeding episode prevention, including doses related to anticipated hemostatic challenges such as physical activity.

### Management of Bleeding Episodes After Discontinuation of Fitusiran

Monthly AT follow-up (AT FU) visits are to be conducted after EOS or after discontinuation of fitusiran treatment to monitor AT recovery (AT activity >60% per the central laboratory).

After EOS or discontinuation of fitusiran participants may resume their local standard management of bleeding episodes with CFC or BPAs once their AT activity levels return to at least 60% (per the central laboratory). Prior to AT recovery the bleed management dosing guidelines should be followed.

### Example 2: Model and Simulation Analysis to Determine Fitusiran Prophylaxis Treatment Commencement and Dose Level

For patients who have received emicizumab, it is not possible to detect the hemostatic effect of emicizumab using standard coagulation assays. Per the manufacturer's guidance, the effect of emicizumab can be present for up to six months after the last dose. The study is intended to describe safety when initiating fitusiran when residual emicizumab is present after approximately two months of the emicizumab dosing cessation period. The residual level from the last emicizumab dose will still confer a significant level of protection against spontaneous bleeding after approximately two months even if a suboptimal prevention of bleeding can be expected during the second month after cessation (see, e.g., Chuansumrit et al., Blood (2021) 138:2116).

The scientific rationale for the switching strategy is based in part on a pharmacokinetics and drug metabolism (PKDM) simulation in a fitusiran Quantitative

Systems Pharmacology (QSP) model developed with thrombin generation (TG) and FVIII-like activity equivalence measures, theoretical concerns, and considerations of good clinical practice. Modelling analysis support the practical approach with a 2- to 2.5-month washout considered appropriate for fitusiran initiation. This washout period is substantially shorter than the current recommended 6-month washout period for emicizumab.

Switching is planned as a cross-tapering-like design with the PD effect from the last dose of emicizumab decreasing gradually while the PD effect of fitusiran is simultaneously built up. The objective and predicted benefit of this strategy is to avoid unacceptable break-in bleeding following the last emicizumab dose while avoiding unnecessary excess coagulation potential after the start of fitusiran.

After a period of approximately two months, fitusiran 20 mg—the lowest dose investigated in adults with a potential to reach AT activity level in a desired range of 15% to 35%—will be initiated at Q2M regimen with the aim for gradual increase in TG potential while emicizumab residual levels continue to decrease after two months of discontinuation to ensure a sustained TG potential before the next fitusiran dosing and consolidate protection against bleeding after the introduction of fitusiran.

A modeling and simulation analysis was developed to predict the total FVIII-like activity during the proposed washout period for emicizumab and the initiation of fitusiran treatment. The published emicizumab population PK (popPK) model and reported emicizumab-FVIII equivalency data were analyzed to account for the FVIII-like activity during the washout period. Two complimentary approaches consisting of internally developed population PK/PD (popPK/PD) and QSP models were implemented to predict the fitusiran-derived FVIII-like activity. This analysis involved a popPK/PD model for simulating AT levels under the initiation of 20 mg Q2M regimen of fitusiran and the QSP-based hemostatic equivalency previously established between AT lowering and FVIII-like activity (see, e.g., Kaddi et al., Blood. (2022) 140 (Suppl 1): 5606-7) for predicting FVIII-like activity in severe Hemophilia A participants.

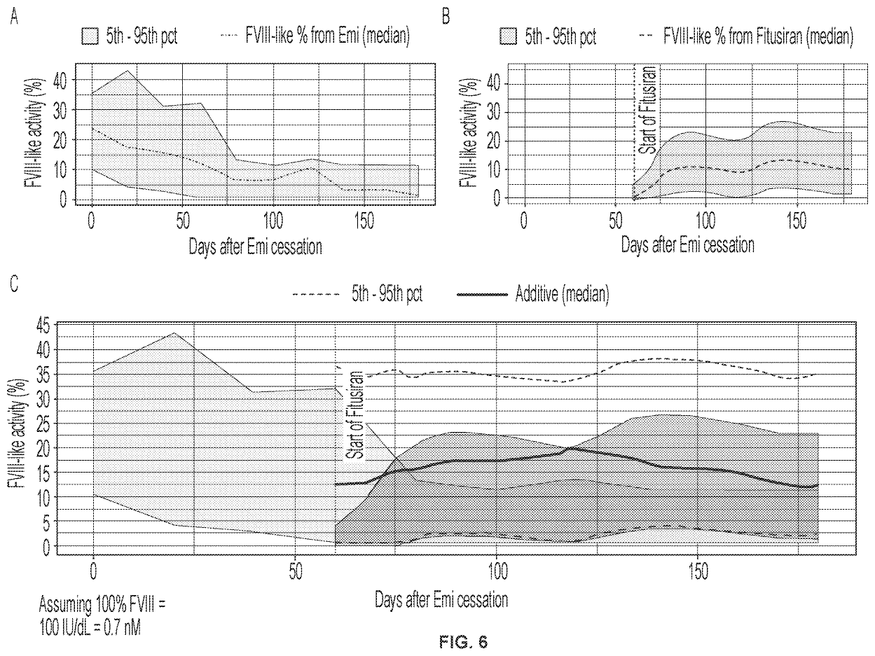

The total FVIII-like activity derived from emicizumab and fitusiran was assumed to be additive. This analysis suggests total FVIII-like activity will remain below 35% FVIII when 20 mg Q2M fitusiran is administered 2 months after the last dose of emicizumab (3 mg/kg QW for four weeks followed by 3 mg/kg Q2M) (FIG. 6).

No escalation in fitusiran dose will happen before four months (i.e., complete wash-out of emicizumab). In addition to the balanced switching strategy and risk mitigation measures, participants will be closely monitored for bleeding occurrence. A bleeding management guidance with reduced doses of factor/BPAs and regular appropriate laboratory testing will be implemented. Treatment of breakthrough bleeds with aPCC will be allowed only after the complete emicizumab clearance.

All PK/PD modeling described below as conducted with the objective of finding dosing regimens that maintain AT activity levels between 15% and 35% as a risk mitigation strategy for vascular thrombotic events.

A population pharmacokinetic/pharmacodynamic (pop PK/PD) model that describes the dynamics of AT activity level for participants treated with fitusiran was developed to find dosing regimens that could maintain AT activity levels between 15% and 35% as a risk mitigation strategy for vascular thrombotic events. The model was characterized on AT activity data from clinical studies. The model with the estimated parameters was used to simulate AT activity level at steady state for different dosing scenarios in a virtual population of 1000 participants.

The results of the simulations are shown in Table 5. Every two months (Q2M) and every month (QM) refer to fitusiran dosing every eight weeks and every four weeks, respectively; TroughAT and PeakAT are trough and peak AT activities; 5th, median and 95th refer to 5th percentile, median and 95th percentile, respectively.

TABLE 5
Simulated AT Activity Percentiles For Various Doses of Fitusiran

| Dose and | | SS TroughAT | | | SS PeakAT | | | |
|---|---|---|---|---|---|---|---|---|
| Regimen | minAT | 5^{th} | Median | 95^{th} | 5^{th} | Median | 95^{th} | maxAT |
| 80 mg QM | 5.28 | 6.63 | 9.31 | 14.49 | 6.82 | 9.71 | 15.42 | 27.39 |
| 50 mg QM | 5.88 | 7.69 | 11.53 | 19.03 | 7.92 | 12.15 | 20.4 | 36.45 |
| 50 mg Q2M | 6.95 | 9.68 | 15.57 | 26.38 | 11.85 | 21.09 | 38.11 | 64.38 |
| 20 mg QM | 7.63 | 11.42 | 19.28 | 33.37 | 12.01 | 20.59 | 35.49 | 58.92 |
| 20 mg Q2M | 9.76 | 15.71 | 27.15 | 44.65 | 20.25 | 36.66 | 59.24 | 90.15 |

Abbreviations: AT: antithrombin; max: maximum; QM: once monthly; Q2M: every two months; SS: steady state.

The starting dose for all participants in this study will be 20 mg Q2M. Four months after the first dose of fitusiran, participants who have either AT levels greater than 35% or who require escalation for clinical reasons will be eligible to escalate to 50 mg Q2M. Based on the simulation results, at the 50 mg Q2M regimen, approximately 45.7% of the virtual participants are expected to have AT activity <15% and therefore may require a de-escalation in dose. Per protocol, these participants will need to de-escalate from 50 mg Q2M to 20 mg Q2M.

The simulations for lower dosing regimens in 1000 de-novo virtual participants, namely 20 mg Q2M and 20 mg QM, predict that 3.8% and 23.4% of the participants, respectively, would have AT activity level <15%. Thus, based on the simulations, 20 mg Q2M was considered an appropriate de-escalation regimen for the 45.7% participants who may have AT activity level <15% at 50 mg Q2M. On de-escalation to 20 mg Q2M, approximately 8% of the de-escalated participants may have AT activity level <15% and approximately 7% of the de-escalated participants may have AT activity level >35%, while the rest, 84% to 85%, of de-escalated participants are predicted to have AT activity level within the target AT window of 15% to 35%.

1. A method of routine prophylaxis to prevent or reduce the frequency of bleeding episodes in a human patient having hemophilia A who has been on prophylactic treatment with emicizumab, comprising subcutaneously administering to the human patient in need thereof
a first dose of fitusiran between about one and about six months after termination of the prophylactical treatment with emicizumab, optionally wherein the first dose of fitusiran is in the amount of about 10 or about 20 mg, and
subsequent doses of fitusiran in the amount of about 10-80 mg about once a month (QM) or about every four weeks (Q4W), or about once every other month (Q2M) or about once every eight weeks (Q8W).

2. A method of maintaining an appropriate thrombin generation (AG) potential in a human patient having hemophilia A with or without inhibitors who has been on prophylactic treatment with emicizumab, comprising subcutaneously administering to the human patient in need thereof
a first dose of fitusiran between about one and about six months after termination of the prophylactical treatment with emicizumab, optionally wherein the first dose of fitusiran is in the amount of about 10 or about 20 mg, and
subsequent doses of fitusiran in the amount of about 10-80 mg about once a month (QM) or about every four weeks (Q4W), or about once every other month (Q2M) or about once every eight weeks (Q8W).

3. The method of claim 1 or 2, wherein the patient is a hemophilia A patient with inhibitors.

4. The method of claim 1 or 2, wherein the patient is a hemophilia A patient without inhibitors.

5. The method of any one of claims 1-4, wherein the patient has been on prophylactic treatment with emicizumab at a dose of about 1.5 mg/kg to about 6 mg/kg about once a week (QW) to about Q4W, or about QM.

6. The method of claim 5, wherein the patient has been on prophylactic treatment with emicizumab at a dose of about 1.5 mg/kg about QW, or about 3 mg/kg about once every other week (Q2W).

7. The method of claim 6, wherein the prophylactic emicizumab treatment in the patient is terminated about two months before the first dose of fitusiran.

8. The method of claim 5, wherein the patient has been on prophylactic treatment with emicizumab at a dose of about 6 mg/kg about Q4W or about QM.

9. The method of claim 8, wherein the prophylactic emicizumab treatment in the patient is terminated about two and a half months before the first dose of fitusiran.

10. The method of any one of claims 1-9, wherein the patient is an adolescent or adult patient, and the first dose and second dose of fitusiran are each about 20 mg and given about two months or about eight weeks apart.

11. The method of any one of claims 1-9, wherein the patient is a pediatric patient, and the first dose and second dose of fitusiran are each about 10 mg and given about two months or about eight weeks apart.

12. The method of any one of claims 1-11, further comprising:
obtaining a measurement of an antithrombin (AT) level after two doses of fitusiran or at a steady state (SS) in the patient; and
performing one of the following steps:
(i) if the AT level is 15-35%, repeating administration of fitusiran at the amount of the first dose about Q2M or about every eight weeks (Q8W);
(ii) if the AT level is >35%, subcutaneously administering to the patient fitusiran at about 50 mg about Q2M or Q8W, or
(iii) if the AT level is <15%, discontinuing or pausing fitusiran treatment.

13. The method of claim 12, comprising, after step (ii),
(a) subcutaneously administering to the patient fitusiran at about 50 mg about QM or about Q4W if the patient has two SS measurements of AT level that are >35%, and then
(a1) subcutaneously administering to the patient fitusiran at 80 mg about QM or about Q4W if the patient has two SS measurements of AT level that are >35%, and de-escalating to the preceding dosing regimen if the patient has more than one measurement of AT level that is <15%; or
(a2) de-escalating to the dosing regimen of (ii) if the patient has more than one measurement of AT level that is <15%;
(b) subcutaneously administering to the patient fitusiran about 20 mg if the patient has more than one measurement of AT level that is <15%, and then
(b1) repeating administration of fitusiran at about 20 mg QM when the patient has a measurement of AT level that is ≥22%; and
(b2) discontinuing fitusiran treatment if the patient has more than one measurement of AT level that is <15%.

14. The method of any one of claims 1-13, further comprising administering an effective amount of a replacement factor VIII or bypassing agent (BPA) to the patient to treat a bleeding episode that occurs seven or fewer days after the first fitusiran dose, wherein the effective amount of the replacement factor or BPA is administered according to the patient's prior dosing regimen of replacement factor or BPA.

15. The method of any one of claims 1-13, further comprising administering an effective amount of replacement factor VIII or bypassing agent (BPA) to the patient to treat a bleeding episode that occurs eight or more days after the first fitusiran dose, wherein the effective amount of the replacement factor or BPA is reduced as compared to the recommended effective amount of the replacement factor or BPA for patients who are not on fitusiran therapy.

16. The method of claim 15, wherein the patient is a patient without factor VIII inhibitors, and wherein the replacement factor is factor VIII and a single dose of factor VIII is no more than 20 IU/kg and optionally is 10 IU/kg, optionally wherein the factor VIII administration is repeated, if needed, in no less than 24 hours.

17. The method of claim 15, wherein the patient is a patient with factor VIII inhibitors, and wherein the BPA is activated prothrombin complex concentrate (aPCC) and a single dose of aPCC is no more than 50 U/kg and optionally is 30 U/kg, optionally wherein the aPCC administration is repeated, if needed, in no less than 24 hours.

18. The method of claim 17, wherein aPCC is administered to treat a bleeding episode no sooner than six months after the last dose of emicizumab or no sooner than four months after the first dose of fitusiran.

19. The method of claim 14 or 15, wherein the patient is a patient with factor VIII inhibitors, and wherein the BPA is recombinant factor VIIa (rFVIIa) and a single dose of rFVIIa is no more than 45 μg/kg, optionally wherein the rFVIIa administration is repeated, if needed, in no less than two hours.

20. Fitusiran for use in a method of any one of claims 1-19.

21. Use of fitusiran in the manufacture of a medicament for use in a method of any one of claims 1-19.

22. Fitusiran for use in routine prophylaxis to prevent or reduce the frequency of bleeding episodes characterized in that
the human patient having hemophilia A has been on prophylactic treatment with emicizumab and
comprising subcutaneously administering to the human patient in need thereof
a first dose of fitusiran between one and six months after termination of the prophylactical treatment with emicizumab, optionally wherein the first dose of fitusiran is in the amount of 10 or 20 mg, and
subsequent doses of fitusiran in the amount of 10-80 mg once a month (QM) or every four weeks (Q4W), or once every other month (Q2M) or once every eight weeks (Q8W).

23. Fitusiran for use in treatment of hemophilia A characterized in that
the treatment comprises routine prophylaxis to prevent or reduce the frequency of bleeding episodes,
the human patient having hemophilia A has been on prophylactic treatment with emicizumab and
comprising subcutaneously administering to the human patient in need thereof
a first dose of fitusiran between one and six months after termination of the prophylactical treatment with emicizumab, optionally wherein the first dose of fitusiran is in the amount of 10 or 20 mg, and
subsequent doses of fitusiran in the amount of 10-80 mg once a month (QM) or every four weeks (Q4W), or once every other month (Q2M) or once every eight weeks (Q8W).

24. Fitusiran for use in the treatment of hemophilia A with or without inhibitors characterized in that
the treatment comprises maintaining an appropriate thrombin generation (AG) potential,
the human patient having hemophilia A has been on prophylactic treatment with emicizumab and
comprising subcutaneously administering to the human patient in need thereof
a first dose of fitusiran between one and six months after termination of the prophylactical treatment with emicizumab, optionally wherein the first dose of fitusiran is in the amount of 10 or 20 mg, and
subsequent doses of fitusiran in the amount of 10-80 mg once a month (QM) or every four weeks (Q4W), or once every other month (Q2M) or once every eight weeks (Q8W).
